(19) United States
(12) Patent Application Publication
(10) Pub. No.: US 2020/0080052 A1
(43) Pub. Date: Mar. 12, 2020

(54) METHOD OF REDUCING CHROMOSOME SEGREGATION ERROR IN CELLS OF THE EARLY EMBRYO

(71) Applicant: CENTRE HOSPITALIER DE L'UNIVERSITÉ DE MONTRÉAL, Montreal (CA)

(72) Inventors: DAVID GREG FITZHARRIS, MONTREAL (CA); CAYETANA VAZQUEZ DE CASTRO DIEZ, BARCELONA (ES)

(21) Appl. No.: 16/561,296

(22) Filed: Sep. 5, 2019

Related U.S. Application Data
(60) Provisional application No. 62/729,090, filed on Sep. 10, 2018.

(51) Int. Cl. C12N 5/073

(52) U.S. Cl. CPC C12N 5/0604; C12N 2501/40

(57) ABSTRACT

The present disclosure presents a method for reducing chromosome segregation errors in cultured cells of the early embryo by contacting the cells with an effective dose of an inhibitor of the anaphase promoting complex (APC/C) that directly binds to the APC/C. It also provides cells of the early embryo so produced and kits comprising the inhibitor.

### CROSS REFERENCE TO RELATED APPLICATIONS

This application claims benefit, under 35 U.S.C. § 119(e), of U.S. provisional application Ser. No. 62/729,090, filed on Sep. 10, 2019. The above-document is incorporated herein in its entirety by reference.

### STATEMENT REGARDING FEDERALLY SPONSORED RESEARCH OR DEVELOPMENT

N.A.

### FIELD OF THE DISCLOSURE

The present disclosure relates to a method of reducing chromosome segregation error in cells of the early embryo. More specifically, the present disclosure is concerned with a method of reducing chromosome segregation error in cells of the early embryo using an inhibitor that directly binds to APC/C.

### REFERENCE TO SEQUENCE LISTING

N.A.

### BACKGROUND OF THE DISCLOSURE

The goal of cell division is to accurately share replicated chromosomes between newly forming daughter cells. Errors result in cells with erroneous numbers of chromosomes, termed aneuploidy. The early mammalian embryo is a notable scenario where aneuploidy is common. As a result of segregation errors during the first several mitoses after fertilisation, preimplantation embryos are often mosaic, with both euploid and aneuploid cells, which reduces the potential of a successful pregnancy Vanneste et al., 2009, Vázquez-Diez et al., 2018). However, the underlying cellular cause for the inherent tendency of embryonic blastomeres to mis-segregate chromosomes is unknown.

The major cellular safeguard preventing segregation errors is the spindle assembly checkpoint (SAC), a near-ubiquitous signalling pathway that delays anaphase onset until chromosomes are correctly aligned with kinetochores correctly attached to spindle microtubules. Mis-attached kinetochores catalyse generation of Mitotic Checkpoint Complex (MCC), a diffusible signal that inhibits the anaphase promoting complex (APC/C), delaying anaphase until correct attachment is achieved (Lara-Gonzalez et al., 2012; Musacchio et al., 2015). Whilst embryo aneuploidy can be exacerbated by SAC inhibition (Bolton et al., 2016; Wei et al., 2011), a detailed examination of SAC function, or an explanation as to why segregation errors are normally common in the early embryo, is yet to be presented. SAC function has been well studied in mammalian oocytes, where it is relatively inefficient in preventing segregation errors (Gui et al., 2012; Lane et al., 2017; Kolano et al., 2012; Nagaoka et al., 2012). This is likely due in part to the need for limited amounts of diffusible MCC to inhibit APC/C throughout the cytoplasm in an extraordinarily large cell (Kyogoku et al., 2017; Lane et al., 2017). Consistently, in C. elegans embryos the SAC is ineffective in early embryos and becomes more effective as cells reduce in size during early development (Galli et al., 2016; Gerhold et al., 2018).

### SUMMARY OF THE DISCLOSURE

In the present disclosure, the inventors used live imaging, micromanipulation, gene knockdown, and pharmacological approaches in mouse embryos to appraise SAC function in in the early mammalian embryo. The inventors show that SAC signalling operates in embryos and limits the number of segregation errors but that cells frequently enter anaphase despite severely misaligned chromosomes with SAC-active kinetochores. While the inventors tested the hypothesis that large cytoplasmic volume hampers the checkpoint in the very earliest stages of development until smaller cell sizes are attained, they unexpectedly discovered that frequent segregation error in the mouse embryo and failure to delay anaphase is not attributable to the large size of early embryo cell but is permitted by a failure of SAC-active kinetochores to prevent anaphase, that is not related to cytoplasmic volume. The present disclosure shows that mildly inhibiting the APC/C can prolong mitosis and thereby increase the probability of correct alignment being achieved before the completion of cell division, and thus lower the number of segregation errors. The present disclosure shows that modulation of the SAC-APC/C axis increases the likelihood of faithful chromosome segregation in cultured embryos.

More specifically, in accordance with the present disclosure, there is provided the following items:

Item 1. Method for reducing chromosome segregation errors in cultured cells of the early embryo by contacting the cells with an effective dose of an inhibitor of the anaphase promoting complex (APC/C) that directly binds to the APC/C.

Item 2. The method of item 1, wherein the inhibitor is proTAME or TAME.

Item 3. The method of item 1 or 2, wherein the effective dose is a nanomolar amount.

Item 4. The method of item 1 or 2, wherein the effective dose is about 0.001 nM to about 500 nM.

Item 5. The method of any one of items 1 to 3, wherein the effective dose is about 0.01 nM to about 100 nM.

Item 6. The method of any one of items 1 to 4, wherein the cells are at the 2-cell stage to blastocyst stage.

Item 7. The method of any one of items 1 to 5, wherein the inhibitor increases the duration of mitosis, reduces the number of micronucleis per cell, reduces the incidence misaligned chromosomes at anaphase onset or a combination thereof.

Item 8. The method of any one of items 1 to 7, wherein the inhibitor does not influence blastocyst cell number as compared to cells of the early embryo not contacting with the inhibitor.

Item 9. The method of any one of items 1 to 8, wherein the cells are mammalian cells.

Item 10. The method of item 9, wherein the cells are human cells.

Item 11. Cells of the early embryo obtained by the method defined in any one of items 1 to 10.

Item 12. Composition comprising the cells of the early embryo defined in item 11.

Item 13. A kit for reducing chromosome segregation errors in cultured cells of the early embryo comprising (a) an inhibitor of the anaphase promoting complex (APC/C) that directly binds to the APC/C; and (b) (i) instructions to use the inhibitor for reducing chromosome segregation errors in cultured cells of the early embryo; (ii) cells of the early embryo; and (iii) a combination of (i) and (ii).

### Definitions

The present disclosure provides a method for reducing chromosome segregation errors in cultured cells of the early embryo by contacting the cells with an effective dose of an inhibitor of the anaphase promoting complex (APC/C), wherein the inhibitor binds directly to the APC/C.

The present invention therefore relates to a method that can result in an embryo with reduced aneuploidy (as compared to when no inhibitor of the present invention is used) and improve embryo development and survival. This method can be used to improve the success of assisted reproduction, in vitro fertilization and in the generation of transgenic animals.

The present invention encompasses APC/C inhibitors which exercise their inhibiting activity by directly binding to APC/C. In a specific embodiment, by such binding, the inhibitor prevents the APC/C activators such as Cdc20 and Cdh1 to interact with and/or activate APC/C. Without being so limited, such inhibitors include proTAME, and TAME.

As used herein, the term “reduces” or “reducing” (in the context of e.g., reducing chromosome segregation errors such as but not limited to reducing the number of micronucleis per cell; reducing the incidence misaligned chromosomes at anaphase onset; reducing kinetochore-microtubule misattachment; reducing chromosome non-disjunction; reducing chromosome breakage; and/or reducing chromosome bridges) refers to a reduction of at least 10% as compared to a control (e.g., control embryotic cells not treated with an inhibitor of the present disclosure), in an embodiment of at least 20% lower, in a further embodiment of at least 30% lower, in a further embodiment of at least 40% lower, in a further embodiment of at least 50% lower, in a further embodiment of at least 60% lower, in a further embodiment of at least 70% lower, in a further embodiment of at least 80% lower, in a further embodiment of at least 90% lower, in a further embodiment of 100% (complete inhibition).

Similarly, as used herein, the term “increases” or “increasing” (in the context of e.g., increasing the duration of mitosis) refers to an increase of at least 10% as compared to a control (e.g., control embryotic cells not treated with an inhibitor of the present disclosure), in an embodiment of at least 20% higher, in a further embodiment of at least 30% higher, in a further embodiment of at least 40% higher, in a further embodiment of at least 50% higher, in a further embodiment of at least 60% higher, in a further embodiment of at least 70% higher, in a further embodiment of at least 80% higher, in a further embodiment of at least 90% higher, in a further embodiment of 100% higher, in a further embodiment of 200% higher, etc.

As used herein, the term “cultured” in the context of “cultured cells of the early embryo” refers to cells of the early embryo grown in conditions suitable to maintain their function and viability. Without being so limited, such culture conditions include ibidi micro-insert wells mounted on a glass-bottom dish with distilled water providing humidified CO₂ gas supply and previously described in extensive detail (Vázquez-Diez et al., 2018) or any standard mammalian embryo culture system. For example, an artificial culture medium or in an autologous endometrial coculture (on top of a layer of cells from the woran's own uterine lining) can be used. With artificial culture medium, there can either be the same culture medium throughout the period, or a sequential system can be used, in which the embryo is sequentially placed in different media.

For example, when culturing to the blastocyst stage, one medium may be used for culture to day 3, and a second medium is used for culture thereafter. Single or sequential medium are equally effective for the culture of human embryos to the blastocyst stage. Artificial embryo culture media basically contain glucose, pyruvate, and energy-providing components, and optionally amino acids, nucleotides, vitamins, and cholesterol. Methods to permit dynamic embryo culture with fluid flow and embryo movement are also available. (see also Mantikou et al., 2013).

As used herein the term “cells of the early embryo” refers to the one-cell stage embryo (i.e. fertilized oocyte) up until the blastocyst stage of the embryo. In a specific embodiment, it refers to the two-cells stage up until the blastocyst stage (including all stages in between including the four-cell stage, eight-cell stage, morula stage (e.g., sixteen to thirty-two cell stage) and blastocyst stage (e.g., 64-128 cell stage).

As used herein the term effective dose refers to an amount that effectively reduces chromosome segregation errors without inducing significant toxicity. In a specific embodiment, it refers to a nanomolar dose (i.e. about 0.001 nM to about 900 nM). In another specific embodiment, it refers to a dose of about 0.01 nM to about 500 nM. In another specific embodiment, it refers to a dose of about 0.01 nM to about 100 nM.

The inhibitor may be applied to the cells of the early embryo with at least one carrier and/or other agents able to improve cells of the early embryo function and/or survival. Carriers for use in such context optimally can dissolve the inhibitor (e.g. proTAME). Without being so limited such carriers include DMSO. Without being so limited, such agents include APCin.

The present disclosure also encompasses cells of the early embryo produced using the method of the present disclosure. Such cells advantageously contain less aneuploidy than cells generated in vitro without using an inhibitor of the instant disclosure.

Kits

The present disclosure also relates to a kit for reducing chromosome segregation errors in cultured cells of the early embryo comprising an inhibitor of the anaphase promoting complex (APC/C), wherein the inhibitor binds directly to the APC/C in accordance with the present disclosure (e.g., proTAME); and (i) cultured cells of the early embryo; and/or (ii) instructions to use the kit to reduce chromosome segregation errors in cultured cells of the early embryo. Such kits may further comprise at least one other active agent able to prevent or treat an osteocalcin associated disease or condition, or symptom thereof. In addition, a compartmentalized kit in accordance with the present disclosure includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated, and the agents or solutions of each container can be added in a quantitative fashion from one compartment to another.

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the disclosure and does not pose a limitation on the scope of the disclosure unless otherwise claimed.

No language in the specification should be construed as indicating any non-claimed element as essential to the practice of the disclosure.

Herein, the term “about” has its ordinary meaning. In embodiments, it may mean plus or minus 10% of the numerical value qualified.

Unless otherwise defined, all technical and scientific terms used herein have the same meaning as commonly understood by one of ordinary skill in the art to which this disclosure belongs.

Other objects, advantages and features of the present disclosure will become more apparent upon reading of the following non-restrictive description of specific embodiments thereof, given by way of example only with reference to the accompanying drawings.

### BRIEF DESCRIPTION OF THE DRAWINGS

In the appended drawings:

FIGS. 1A-H: SAC signalling fails to prevent mitotic errors. FIGS. 1A and B: Representative confocal images of H2BRFP-expressing embryos and analysis of chromosome alignment prior to anaphase (n=100 divisions at the 16-32-cell transition). Yellow arrows indicate misaligned chromosomes prior to anaphase onset. FIG. 1C: Representative images of a blastocyst and a micronucleus (indicated by the yellow arrowhead). FIGS. 1D and E: Quantification of cell numbers and micronuclei per cell in DMSO and AZ3146-treated blastocyst embryos (mean±SEM, n=>20 embryos/group, t-test, P<0.05) and Control MO and Mad2 MO blastocysts (mean±SEM, n=>20 embryos/group, t-test, P<0.05). FIG. 1F: Representative images of chromosome segregation dynamics in 16-32C transition of H2B:RFP embryos in control DMSO and 20 μM AZ3146. White arrows indicate pre-anaphase misaligned chromosomes; yellow arrows indicate anaphase lagging chromosome resulting in the formation of a micronucleus. FIG. 1G: Duration of mitosis (mean±SEM) and FIG. 1H: incidence of pre-anaphase misaligned chromosomes in DMSO and 20 μM AZ3146 (n=>40 divisions/group, t-test, P<0.05).

FIGS. 2A-B. Mad2 knockdown with Morpholino Oligonucleotides. FIG. 2A: Representative confocal images and fluorescence intensity quantification of Mad2 immunofluorescence in embryos microinjected with Control and Mad2 morpholino oligonucleotides (MO) (mean and SEM, n=>108 cells/group, t-test, P=<0.0001). FIG. 2B: Polar body extrusion (PBE) of meiosis-I in oocytes microinjected with Control and Mad2 MO, resulting in a characteristic acceleration of meiosis-I as previously reported (Homer et al., 2005; Wassmann et al., 2003).

FIGS. 3A-E. Severe spindle damage induces a robust SAC-mediated mitotic arrest. FIG. 3A: Representative images of embryos before and after a 16-hour exposure to the vehicle (DMSO) or different concentrations of the spindle poison nocodazole. FIG. 3B: Measurements of spindle length in DMSO and nocodazole-treated embryos. (mean and SEM, n=>7 metaphases per group). C: Percentage of prometaphase cells in embryos exposed to different nocodazole concentrations (mean and SEM, n=>20 embryos/group, one-way ANOVA, P<0.05). FIG. 3D: Quantification of the percentage of mitotic cells in embryos treated with DMSO or 100 nM nocodazole and/or 20 μM AZ3146. (mean and SEM, n=>13 embryos/group, one-way ANOVA, P<0.0001). FIG. 3E: Representative images of Mad2 recruitment to metaphase spindles in the presence of different concentrations of nocodazole.

FIGS. 4A-G. SAC-active misaligned chromosomes fail to prevent anaphase. FIG. 4A: Representative images of embryos and metaphase spindles after a 16 hour exposure to GSK933295. White arrowheads indicate misaligned chromosomes FIG. 4B: Mean percentage of metaphases containing severely misaligned chromosomes (mean±SEM, n=>7 metaphases/group). FIG. 4C: Percentage of prometaphase cells in embryos exposed to GSK923295 (mean±SEM, n=129 embryos with n>7 embryos per group). FIG. 4D: Representative confocal images of chromosome segregation dynamics of H2BRFP-expressing embryos in DMSO and 500 nM GSK923295. White and yellow arrowheads indicate misaligned chromosomes prior to and during anaphase, respectively. FIG. 4E: Proportion of divisions initiating anaphase in the presence of at least one severely misaligned chromosome (n>50 divisions/group). FIG. 4F: Representative images of a metaphase with a misaligned chromosome at anaphase onset (denoted by black arrowhead) in the four-to-eight-cell mitosis in H2B:RFP-Mad1:EGFP-expressing embryos. White arrowhead indicates sister chromatid separation. FIG. 4G: Proportion of misaligned chromosomes displaying detectable Mad1:EGFP signal at anaphase onset (n=59 misaligned chromosomes from 17 divisions).

FIG. 5. Anaphase onset is unaffected by the number of misaligned chromosomes. Quantification of NEBD to anaphase time and number of misaligned chromosomes at anaphase onset during the eight- to sixteen-cell division of H2B:RFP-expressing embryos in 500 nM GSK (mean and SEM, n=51 divisions per group, one-way ANOVA, P=0.86). Note both NEBD to metaphase and metaphase to anaphase times are extended in the presence of GSK923295.

FIGS. 6A-B. SAC response to nocodazole in unaffected by CENP-E inhibition. FIGS. 6A and B: Representative confocal images and analysis of percentage of prometaphase cells in fixed embryos following a 16 hr treatment with 1 μM nocodazole±500 nM GSK923295 and immuno-stained with Mad2 (mean and SEM, n=>embryos per group, one-way ANOVA, P<0.0001, Tukey's multiple comparisons test, P=0.981).

FIG. 7. Mad2 is recruited to misaligned chromosomes during CENP-E inhibition. Representative confocal images of Mad2 immunofluorescence in 8-cell embryos treated with 500 nM GSK923295.

FIGS. 8A-B. Mad1:EGFP is recruited to chromosomes following nuclear envelope breakdown. Consecutive time-points of confocal time-lapse imaging of the four- to eight-cell division in H2B:RFP-Mad1:EGFP expressing embryos. FIG. 8A: Mad1:EGFP localises to the nuclear lamina prior to NEBD, is lost from the envelope during NEBD, and forms puncta on chromosome in prometaphase. FIG. 8B: Mad1:EGFP (yellow arrowheads) is lost from chromosomes (white arrowheads) as they become aligned at the metaphase plate.

FIGS. 9A-I. SAC strength is cell-size independent. FIG. 9A: Representative images of H2BRFP-expressing embryos at two-cell (2C), morula (16C) and blastocyst stages. FIG. 9B: Quantification of mitosis duration at the different stages examined in control and 10 nM of nocodazole (mean±SEM, n=>24 divisions/group, t-test p<0.05). FIG. 9C: Representation of the percentage change in mitosis duration and FIG. 9D: Percentage increase in the rate of severe lagging chromosomes in 10 nM nocodazole vs. control at 2C and 16C stages. FIG. 9E: Representative images of cytoplasmic removal technique in two-cell embryos. FIGS. 9F and G: Representative images of two-cell and morula H2B:RFP-GAP43:GFP-expressing embryos, respectively. FIGS. 9H and I: Scatterplot of mitosis duration and cell volume measured at metaphase for individual cells of un-manipulated or with cytoplasmic reduction in both cells in control or 10 nM nocodazole conditions at the 2-4C and 16-32C divisions (n>17 divisions/group, correlation analysis, r=−0.11 and −0.17 at 2-4C, r=0.33 and −0.24 at 16-32C, in Control and 10 nM nocodazole, respectively).

FIGS. 10A-C. SAC strength is unaltered by experimental cell size reduction. FIG. 10A: Diagrammatic representation of the experimental design. Cytoplasmic removal was performed in one cell of two-cell embryos expressing H2B:RFP and GAP43:GFP. FIGS. 10B and C: Analysis of cell volume measured at metaphase, and mitosis duration in control or 10 nM nocodazole conditions, at the two-cell and morula-stages, respectively (n=>24 divisions per group).

FIGS. 11A-F. Mild APC/C inhibition reduces chromosome segregation errors. FIG. 11A: Diagrammatic representation of the experimental design. Two-cell embryos were cultured to blastocyst in DMSO or different concentrations of proTAME, for immunofluorescence analysis, or previously microinjected with H2B:RFP for live cell imaging at morula stage. FIG. 11B: Number of cells in blastocysts treated with proTAME (mean and SEM, n>26 embryos/group) and FIG. 11C: Number of micronuclei per cell in blastocyst exposed to different proTAME concentrations (mean±SEM, n>26 embryos/group, one-way ANOVA, P=0.006, Tukey's multiple comparisons test, asterisks denote P<0.01). FIG. 11D: Representative confocal images of H2B:RFP-expressing embryos in DMSO and 10 nM proTAME. Yellow squares delineate cells zoomed in panels, white arrowheads indicate anaphase onset. FIG. 11E: Quantification of mitosis duration in DMSO and 10 nM proTAME (mean±SEM, n>150 divisions/group, Mann-Whitney test, P<0.05). FIG. 11F: Analysis of incidence of mild and severely misaligned chromosomes at anaphase onset in DMSO and 10 nM proTAME (n>150 divisions/group, Fischers exact test, p<0.05).

FIG. 12. Effect of proTAME on blastocyst development. Scoring of developmental stages of embryos at E4.5 following culture in various concentrations of proTAME from the two-cell stage (n=>17 embryos per group).

FIG. 13. Number of cells in blastocysts treated with APCin (mean and SEM).

### DESCRIPTION OF ILLUSTRATIVE EMBODIMENTS

The present disclosure is illustrated in further details by the following non-limiting examples.

### Example 1: Material and Methods

Embryo collection, culture and chemical treatments. All experiments were approved by the Centre de Recherche du Centre Hospitalier de l'Université de Montréal (CRCHUM) Comité Institutionnel de Protection des Animaux (CIPA). Two-cell embryos were obtained from superovulated 2-3-month-old CD-1 female mice mated with CD-1 males. Embryos were collected in M2 media and cultured in KSOM media drops covered in mineral oil at 37° C. in 5% CO₂. For chemical treatments, media was supplemented with the following compounds: AZ3146 (20 μM), GSK923295 (various concentrations), nocodazole (various concentrations), Cytochalasin B (5 μg/ml), MG132 (25 μM), proTAME (various concentrations). All drug stocks are dissolved in DMSO. For experiments involving proTAME, embryos were cultured without oil in 500 μL of media in 4-well plates.

Cytoplasmic Removal and Microinjection.

All micromanipulations were performed on a Leica™ DM14000 inverted microscope fitted with Narashige micromanipulators. Cytoplasmic aspiration and removal were achieved with hydraulic-controlled glass pipettes with paraffin liquid hydraulic control mounted on a piezo-electric drill, to perforate the zona pellucida, in two-cell embryos treated with 10 μM nocodazole, an anti-mitotic agent, and 5 μg/mL Cytochalasin B, known to block the formation of microfilaments. Embryos where thoroughly washed in over a dozen drops of M2 media prior to transfer and incubation in equilibrated KSOM medium for at least 2 hours prior to imaging to allow them to recover from the cytoplasmic removal procedure. Microinjections were performed as described previously (Nakagawa and FitzHarris, 2016; Vázquez-Diez and FitzHarris, 2018; Vázquez-Diez et al., 2016). cRNA was synthesized using mMessenger Machine kit (Ambion) as previously (Nakagawa et al., 2017; Vázquez-Diez et al., 2018), from the following plasmids; pRN4-H2B:RFP, psC2-GAP43:GFP, MajSatTALE:mClover (pTALYM3B15)(Miyanari et al., 2013).

Live Cell Imaging and Immunofluorescence.

All imaging was performed on a Leica™ SP8 confocal microscope with HyD detectors. For live imaging embryos were placed in a heated top-stage incubator with CO₂ supply and imaged with a 20×0.75 NA objective as previously described (Vázquez-Diez et al., 2016). All imaging was performed in ˜2 μL drops under mineral oil, for experiments involving proTAME, embryos were cultured in Ibidi micro-insert wells mounted on a glass-bottom dish with distilled water and the inventors' setup was modified to provide humidified CO₂ gas supply, previously described in extensive detail (Vázquez-Diez et al., 2018).

Z-stack images of embryos (˜50 μm) with step size 2 μm were obtained at a time interval of 30 sec for experiments concerning H2BRFP only, 2 min for H2B:RFP-GAP43:GFP, and 3 min H2B:RFP-Mad1:EGFP experiments. For live-cell experiments at two-cells, eight cell, morula and blastocyst, imaging was started at ˜48 h, 72 h, 84 h and ˜96 h post-hCG administration and mating, respectively. For immunofluorescence embryos were fixed in 2% PFA in PBS (20 min) and permeabilised in 0.25% Triton™ X-100 in PBS (10 min), for Mad2 immunofluorescence embryos were fixed for 15 min in PHEM buffer containing 2% PFA and 0.05% Triton™ X-100, and permeabilised in 0.05% Triton™ X in PHEM buffer for 15 min. Blocking was performed in 3% bovine serum albumin in PBS, for 1 hr at 37° C. or overnight at 4° C. Primary antibodies used were rabbit anti-Mad2 (1:300), mouse anti-alpha-Tubulin (1:1000), anti-rabbit and anti-mouse AlexaFluor secondaries were used at 1:1000 dilution. Alexa-555-Phalloidin (1:300) and Hoechst (1:1000) were used to visualize F-actin and DNA, respectively. Briefly, immunofluorescence confocal imaging was performed on a Leica™ SP8 microscope using a 63×1.4 NA oil objective lens, using a 1.5 μm optical section and acquiring Z-stacks with a step size of 1.5 μm, described in more detail in (Vázquez-Diez et al., 2018). In fixed-cell experiments involving GSK923295 and nocodazole embryos were exposed from the 8-cell stage, ˜72 h post-hCG and mating for ˜16 h and immediately fixed. Blastocyst immunofluorescence analysis was performed at late blastocyst stage, ˜120 h post-hCG.

Image Analysis and Statistics.

All image analysis was performed using Fiji software. Time-lapse Z-stacks were examined to determine mitosis duration and identify chromosome alignment and segregation errors. Spindle length measurements were performed measuring the distance between spindle poles, using Pythagoras' theorem where spindle poles were located on different Z-slices. Mad2 Immunofluorescence was quantified by subtracting nuclear envelope and background maximum grey values. To calculate cell volume for each cell at metaphase, areas delineated by the cell membrane (GAP43:GFP signal) were manually traced and measured at each Z-slice, the sum of the areas was multiplied by the step size (2 μm). All data analysis was performed using GraphPad™ Prism, statistical test used were accordingly noted in figures, α=0.05.

| | | |
|---|---|---|
| REAGENT or RESOURCE | SOURCE | IDENTIFIER |

Antibodies

| | | |
|---|---|---|
| Rabbit anti-Mad2 | Biolegend | 924601 |
| Mouse anti-beta-Tubulin | Sigma | T4026 |
| Alexa Fluor 488 anti-Rabbit | ThermoFisher | A-11008 |
| Alexa Fluor 488 anti-Mouse | ThermoFisher | A-11029 |
| Alexa Fluor 568 anti-Mouse | ThermoFisher | A-11031 |
| Alexa Fluor 555 Phalloidin | ThermoFisher | A-34005 |
| Hoechst | ThermoFisher | H-2399 |

Chemicals, Peptides, and Recombinant Proteins

| | | |
|---|---|---|
| Pregnant Mare's Serum Gonadotrophin | Genway biotech | GWB-2AE30A |
| Human Chorionic Gonadotrophin | Sigma | CG10 |
| M2 Media | Sigma | M7167 |
| KSOM Embryo Culture Media | Millipore Sigma | MR-020P-5F |
| AZ3146 | Calbiochem | 531976 |
| Nocodazole | Calbiochem | 487928 |
| GSK923295 | Cayman Chemical | 18389 |
| Cytochalasin B | Sigma | C6762 |
| MG132 | Calbiochem | 474790 |
| proTAME | R&D Systems Inc. | I-440-01M |
| APCin | TOCRIS | 5747 |

Critical Commercial Assays

| | | |
|---|---|---|
| mMessage Machine Kit | Ambion | SP6 AM1336, T3 AM1348, T7 AM1344 |

Experimental Models: Organisms/Strains

| | | |
|---|---|---|
| Mouse CD-1 IGS | Charles River Laboratories | — |

Oligonucleotides

| | | |
|---|---|---|
| Control Morpholino Oligonucleotide | Gene tools LLC | 5′-CCTCTTACCTCAGTTACAATTTATA-3′ |
| Mad2 Morpholino Oligonucleotide | Gene tools LLC | 5′-GCTCTCGGGCGAGCTGCTGTGCCAT-3′ |

Recombinant DNA

| | | |
|---|---|---|
| pRNA-H2B:RFP | Gift from Alex McDougall | Sorbonne Universités, UPMC Univ Paris 06, |
| | | CNRS, Laboratoire de Biologie du |
| | | Développement |
| | | de Villefranche-sur-mer (LBDV), |
| | | Observatoire Océanologique, FRANCE |
| GAP43:GFP | Gift from Yojiro Yamanaka | Goodman Cancer Research Centre, |
| | | Department of Human Genetics, McGill |
| | | University, CANADA |
| MajSatTALEmClover | Addgene | 47878 |
| pIVT-Mad1:2xEGFP | Gift from Michael Lampson | Department of Biology, University |
| | | of Pennsylvania, USA |

Software and Algorithms

| | |
|---|---|
| Fiji | Schindelin, J.: Arganda-Carreras, I. & Frise, E. et al. (2012), |
| | “Fiji: an open-source platform for biological-image analysis”, |
| | Nature methods 9(7): 676-682, PMID 22743772, |
| | doi: 10.1038/nmeth.2019 |
| Excel | Microsoft Office Professional Plus Excel 2010 |
| GraphPad Prism | GraphPad Prism version 7.00 for MAC/Windows, |
| | GraphPad Software, La Jolla California USA, |
| | www.graphpad.com |

Other

| | | |
|---|---|---|
| 4-well micro-insert | Ibidi | 80489 |

### Example 2: Sac Signalling Fails to Prevent Chromosome Segregation Errors in Embryos

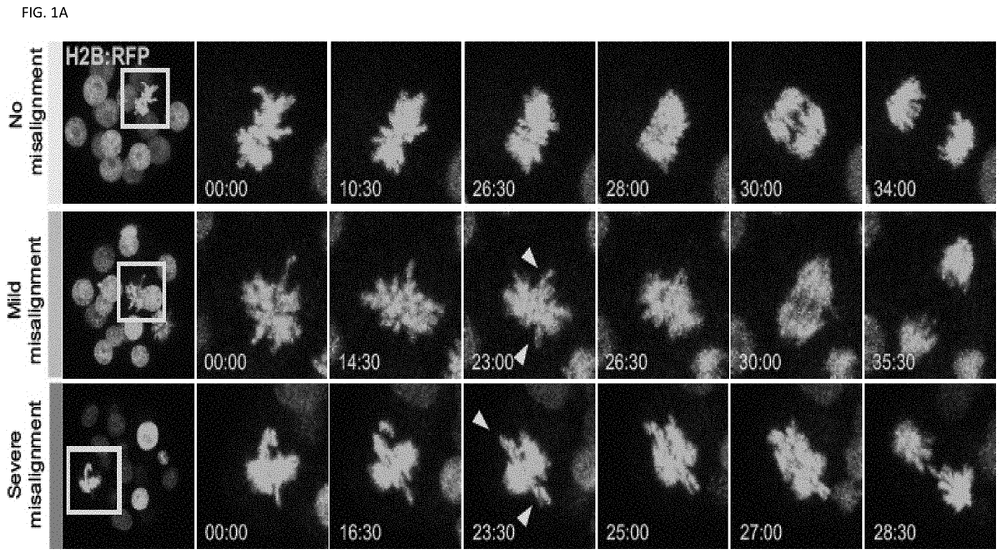

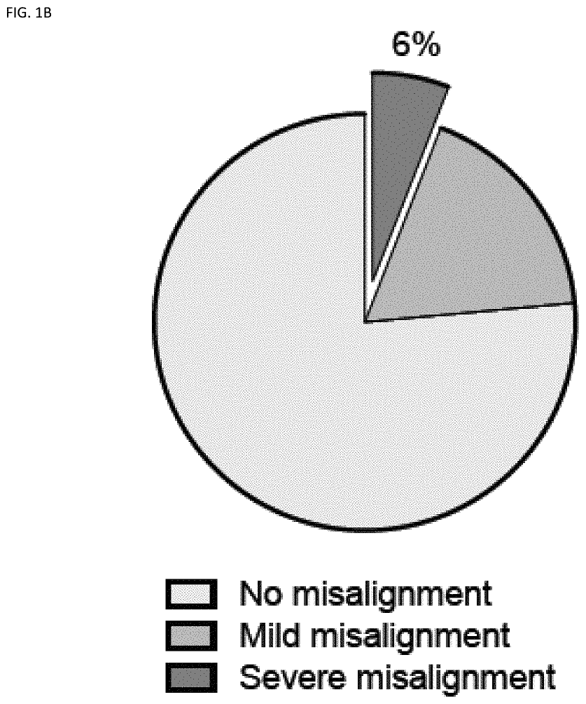

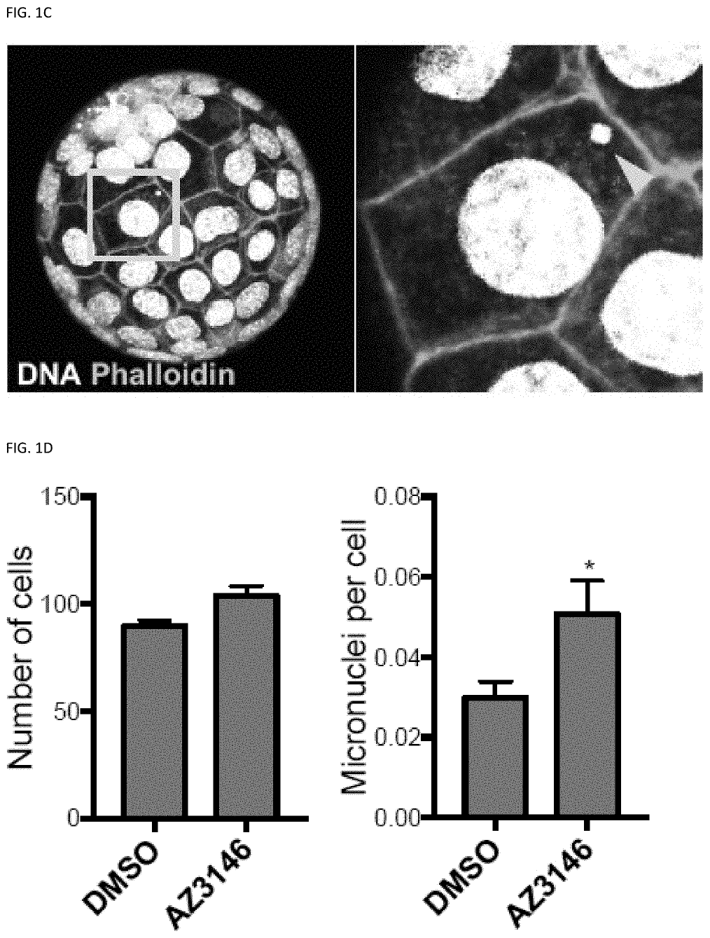

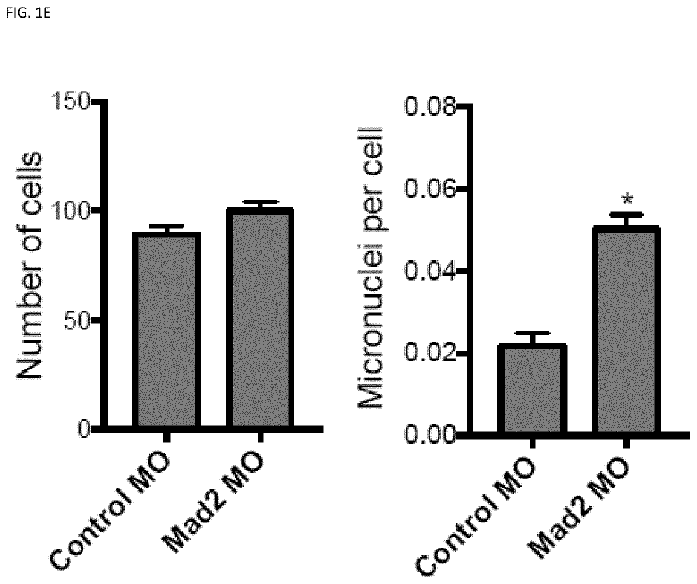

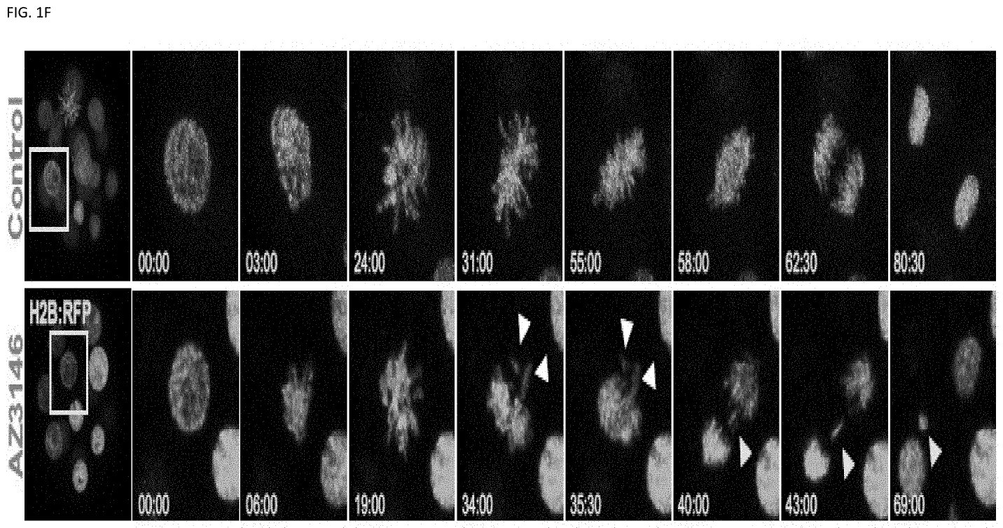

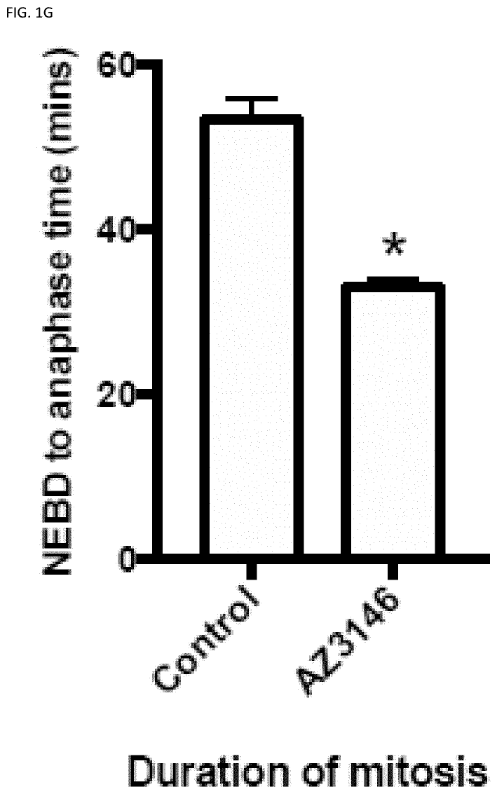

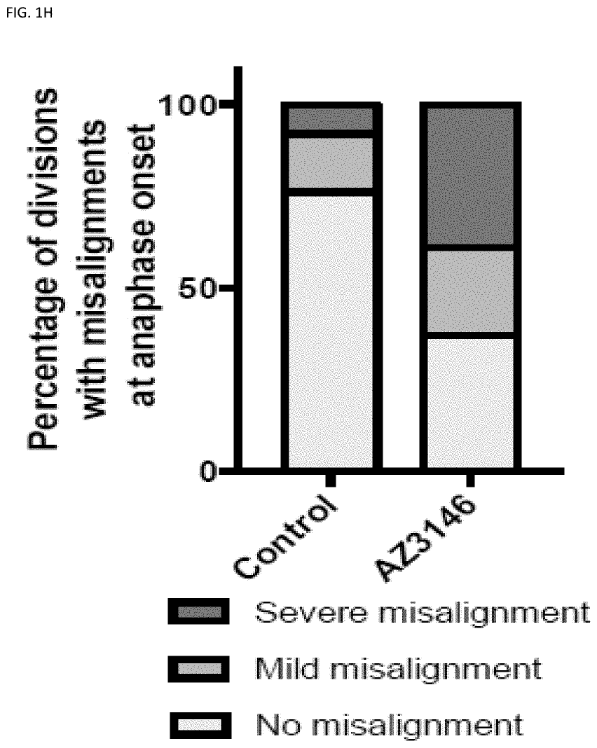

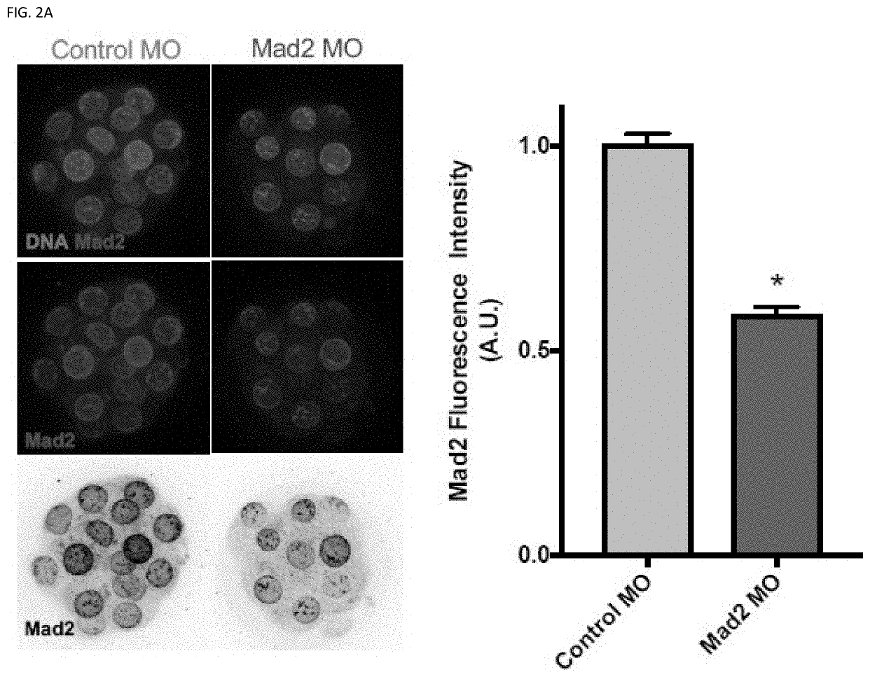

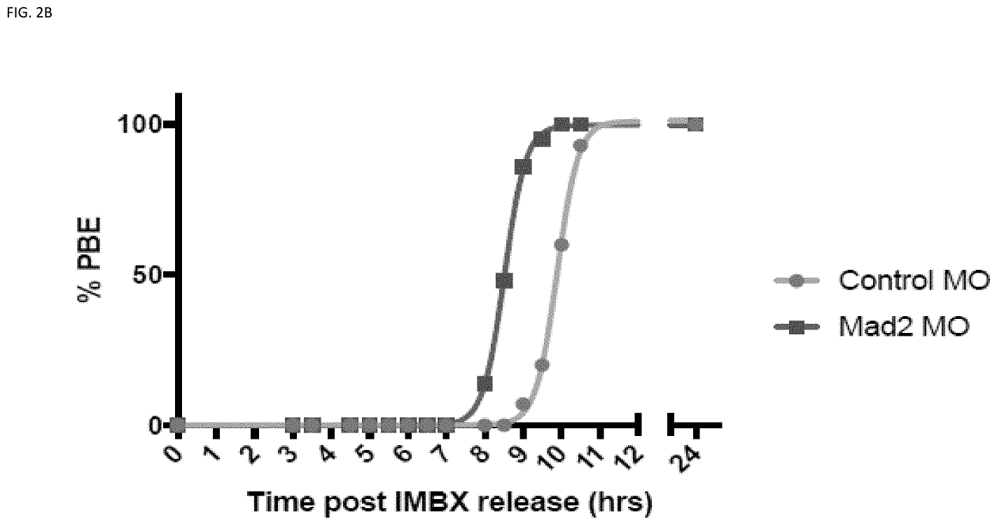

Mouse embryos progress from a fertilised 1-cell embryo to a 16-32 cell stage morula and 64-128 cell blastocyst in 4-5 days, blastomeres approximately halving in size at each division. The inventors first examined chromosome segregation in live H2B:RFP-expressing embryos. Classically in somatic cells, a single misaligned chromosome is sufficient to sustain robust SAC signalling and prevent anaphase (Rieder et al., 1995). In morula-stage embryos, chromosomes were fully aligned at the time of anaphase onset in 76% of divisions, anaphase occurring without any obvious defect. However, a significant subset of divisions progressed into anaphase despite the presence of mildly (18%) or severely (6%) misaligned chromosomes (FIGS. 1A and B). To directly appraise SAC function during normal development, the inventors inhibited the SAC using two complementary approaches; AZ3146, a highly specific inhibitor of Mps1 kinase (Hewitt et al., 2010), and morpholino oligonucleotides (MO) against Mad2 (FIG. 2A). Neither treatment prevented development to blastocyst or altered cell numbers (FIGS. 1C, D and E). Analysis of chromosome segregation dynamics in live embryos revealed that 20 μM AZ3146 shortened mitosis from 53±3 mins to 33±1 mins (P<0.0001), and caused a marked increase in chromosomes remaining misaligned at anaphase onset (63% compared to 24%; P<0.0001) (FIGS. 1F, G and H). The number of micronuclei, which in mouse embryos is a direct indication of accumulated segregation errors (Vázquez-Diez et al., 2016), doubled in AZ3146-treated and Mad2 MO-injected embryos compared to controls (P<0.01; FIGS. 1D and E). Together these data suggest that SAC signalling serves to prolong mitosis in early embryos, thereby allowing more time for complete chromosome alignment prior to anaphase onset. However, blastomeres in normally-cultured embryos nonetheless frequently enter anaphase before chromosome alignment is complete.

### Example 3: Sac-Active Misalignments Fail to Prevent Anaphase

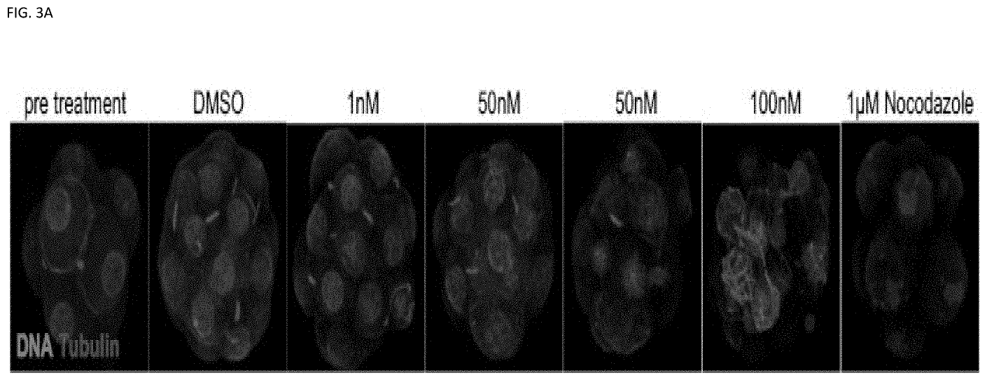

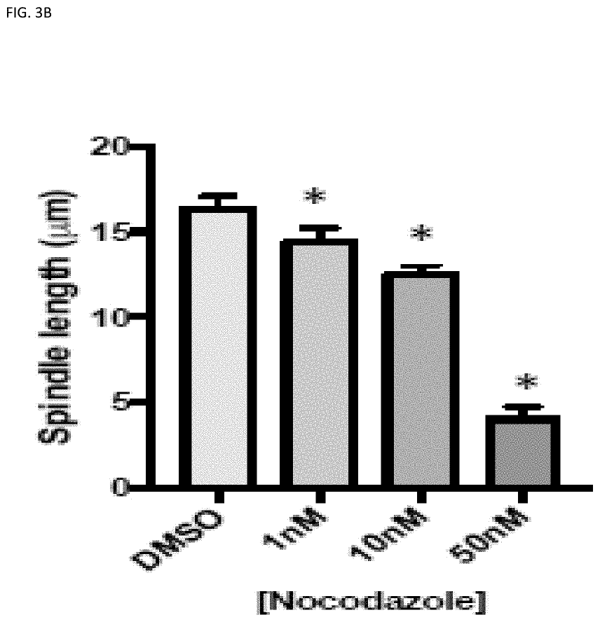

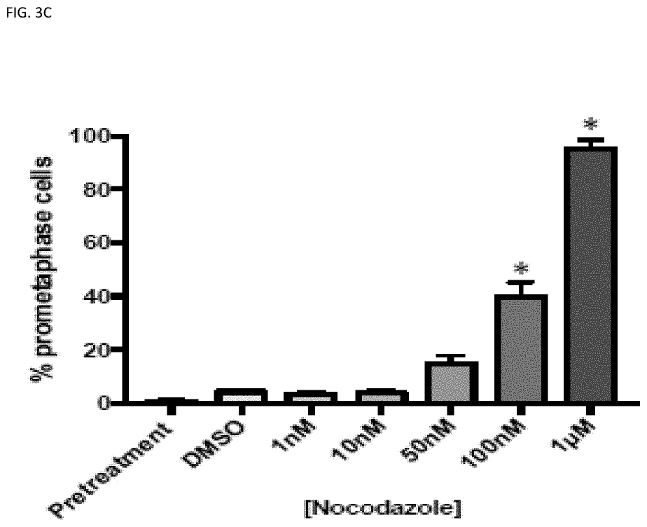

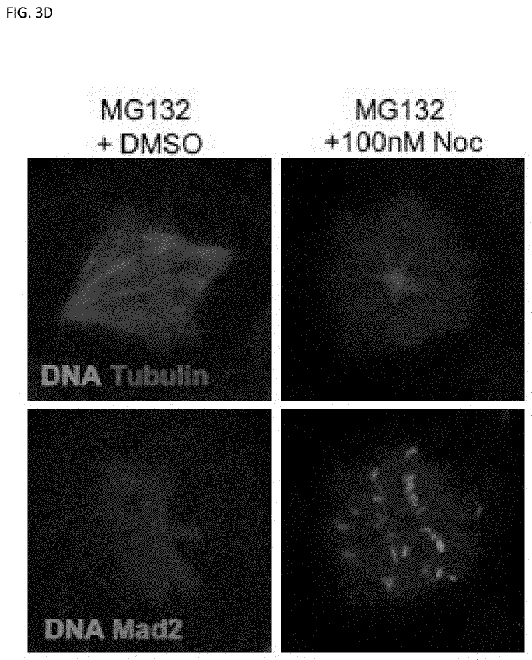

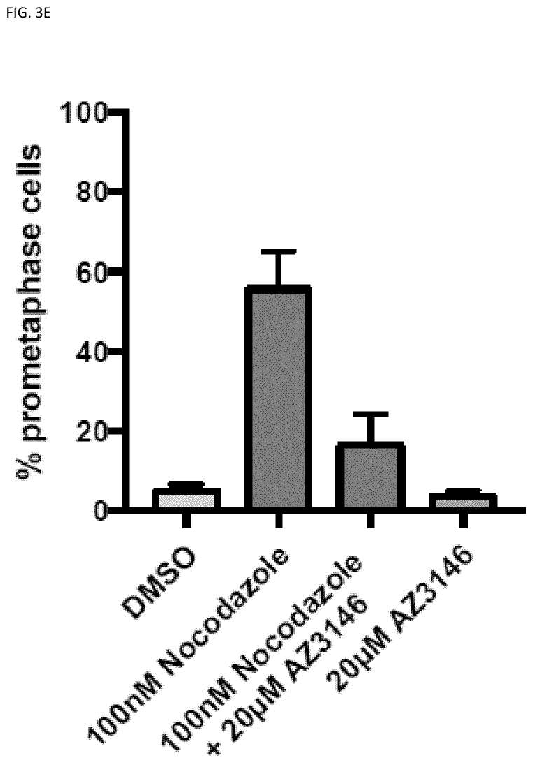

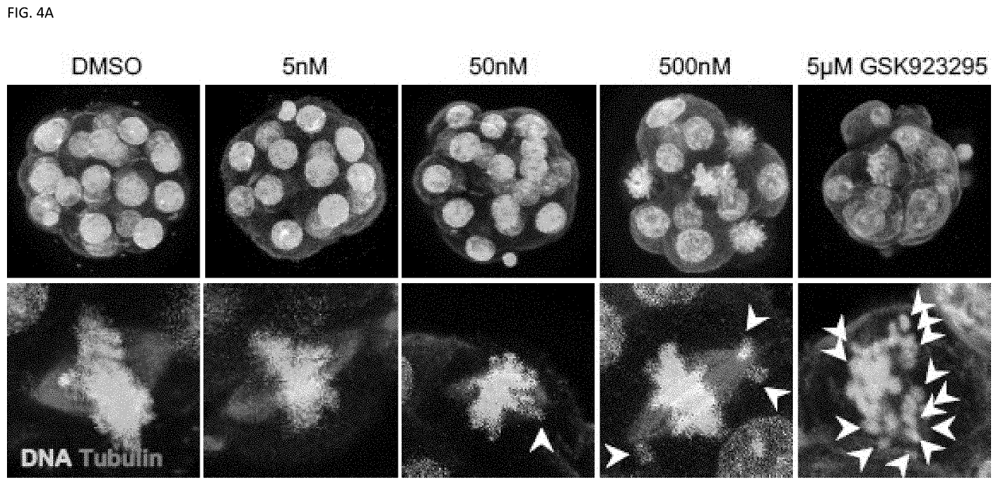

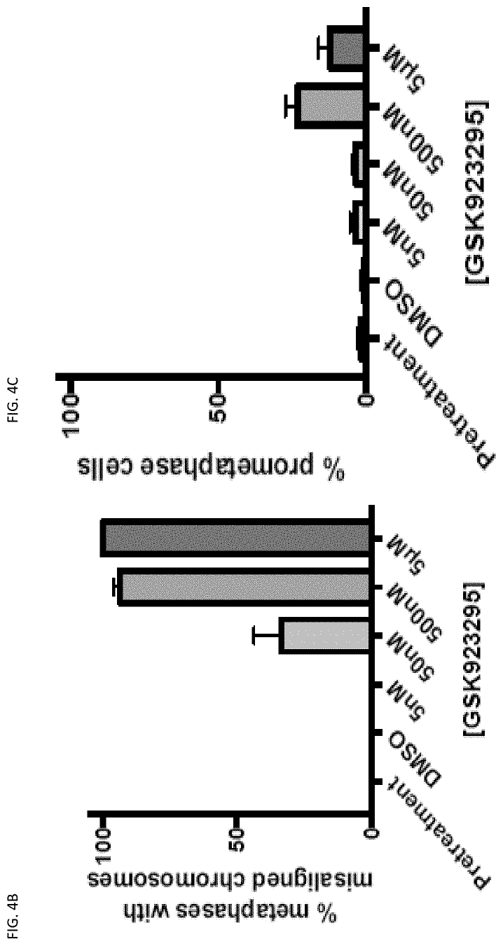

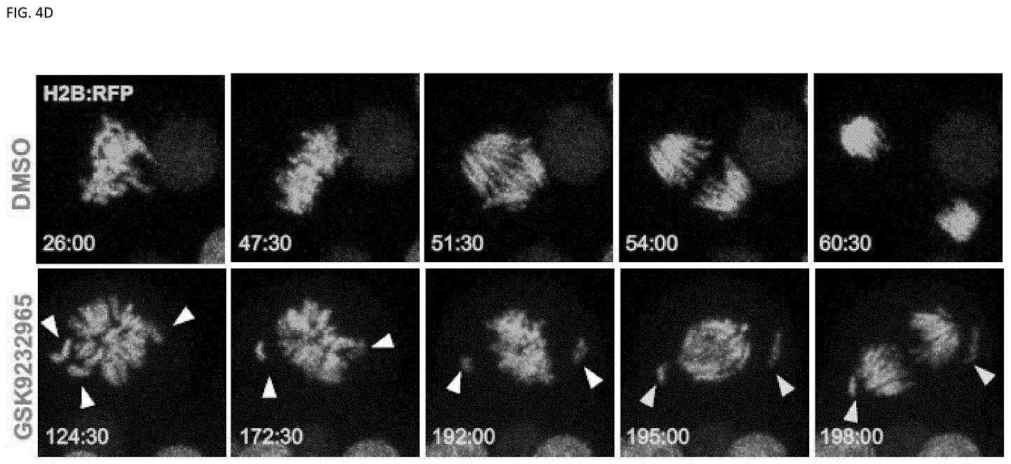

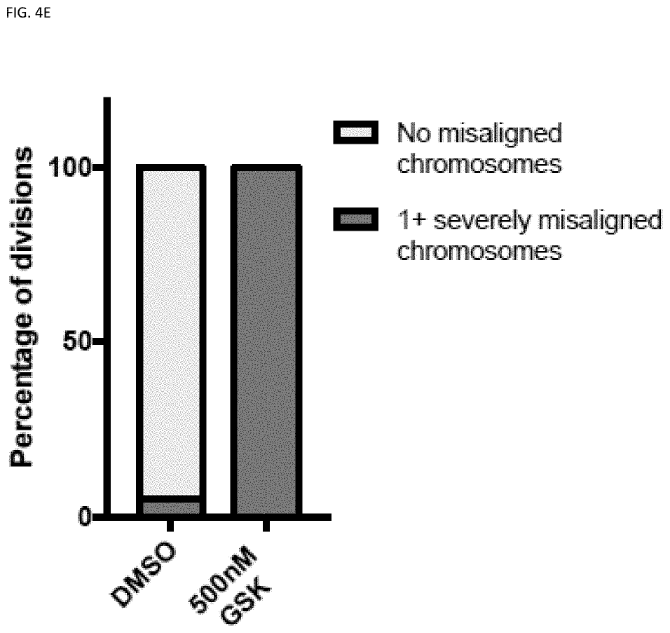

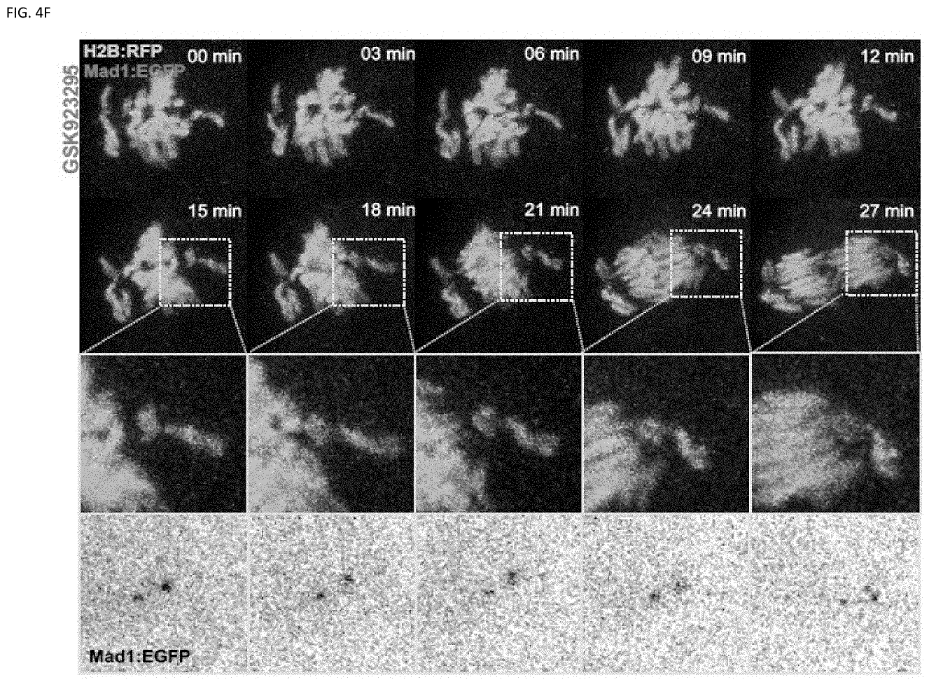

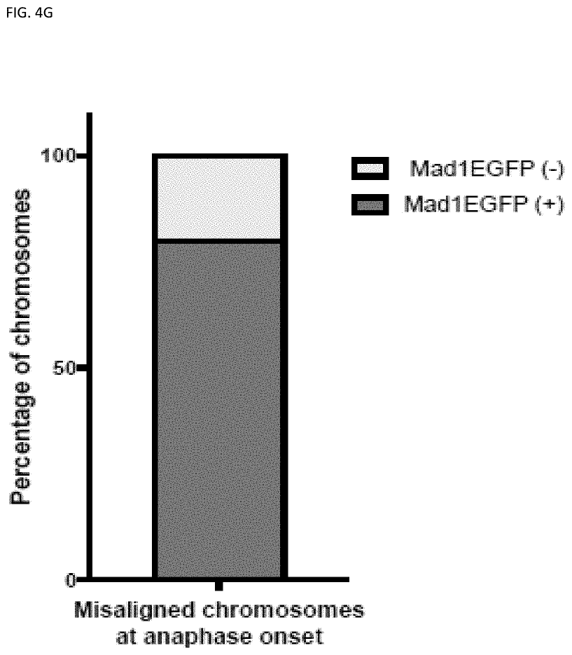

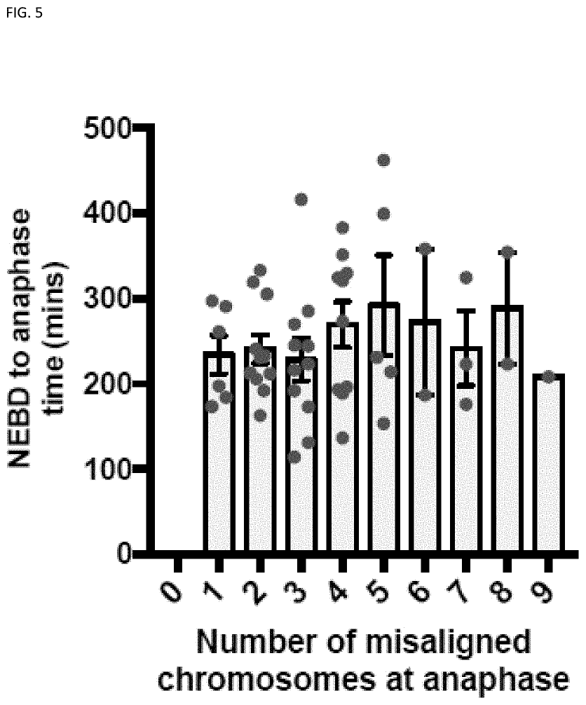

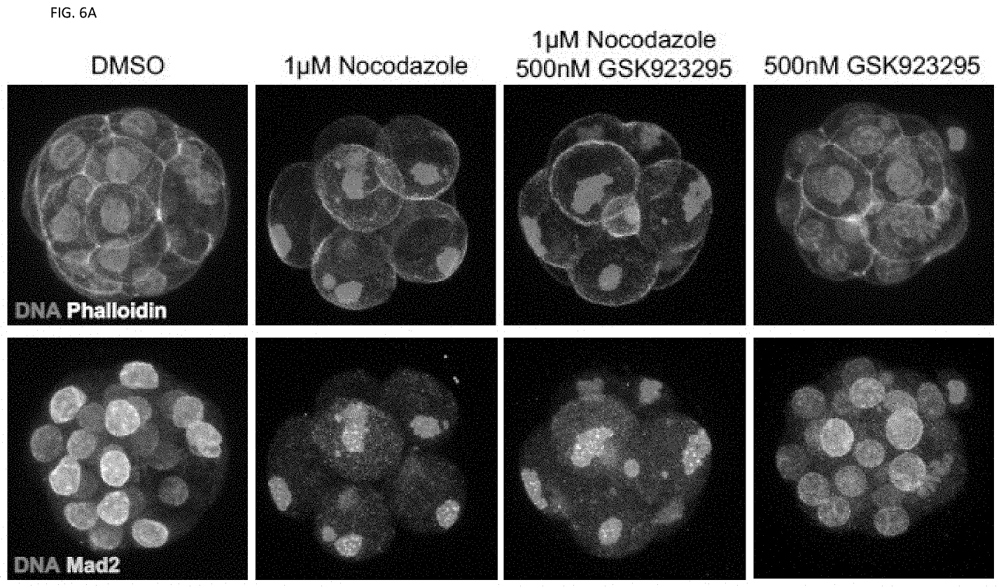

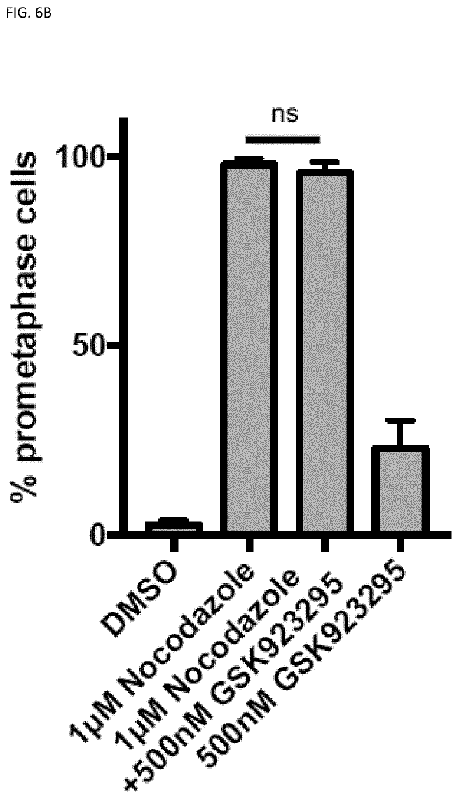

The inventors next tested the ability of the SAC to respond to spindle defects. Treating embryos with the spindle poison nocodazole caused a pronounced mitotic arrest (mitotic index 40% and 95% in 100 nM and 1 μM nocodazole, respectively) (FIGS. 3A, B and C). Mad2 was evident at kinetochores in nocodazole-treated cells, and the arrest was reversible by AZ3146 (FIGS. 3D and E). Thus, severe spindle damage can elicit a classic SAC-dependent mitotic arrest in early embryos, similar to other cell types (Brito et al., 2008; Chen et al., 1996; Meraldi et al., 2004). To more discerningly appraise the SAC's capacity to prevent anaphase, the inventors employed GSK923295, a specific inhibitor of chromokinesin CENP-E, which in mammalian somatic cells prevents chromosome alignment, leading to SAC activation and mitotic arrest (Bennett et al., 2015). Fixed cell analysis revealed that 500 nM and 5 μM of GSK923295 resulted in multiple misaligned chromosomes in almost all metaphase cells in morulae, without obviously disrupting spindle architecture (93% and 100% respectively) (FIGS. 4A and B). However, embryos displayed only a moderate increase in mitotic index (23% and 12% respectively), suggesting a failure of cells to arrest in mitosis (FIG. 4C). Live imaging of the 8-16 cell transition in 500 nM GSK923295 confirmed the failure to arrest in mitosis, and revealed anaphase onset occurred with at least one, and as many as nine severely misaligned chromosomes in all cases (mean of 3.6±0.3 per cell) (FIGS. 4D and E, FIG. 5). Embryos treated simultaneously with nocodazole and GSK923295 mounted a robust mitotic arrest, revealing that GSK923295 had unexpectedly not inactivated the SAC (FIG. S4).

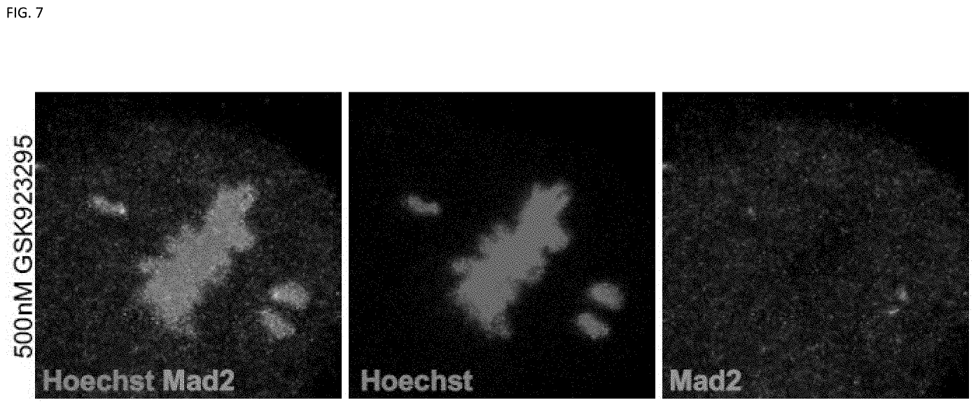

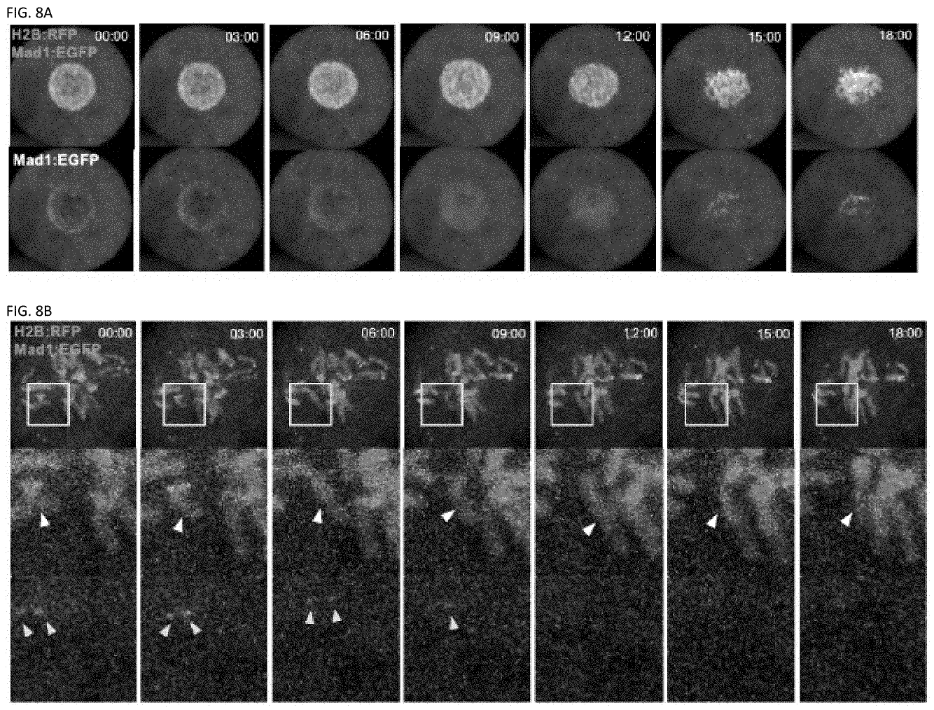

The inventors wondered whether the kinetochores of misaligned chromosomes mount a SAC signal. Fixed-cell analysis showed that Mad2 immunolocalised on kinetochores of misaligned, but not aligned chromosomes in prometaphase (FIG. 7). To determine whether recruitment of SAC components to the kinetochore persists at the time of anaphase onset, the inventors employed Mad1:GFP, an extensively used reporter of SAC activity at kinetochores (Emre et al., 2011; Heinrich et al., 2014; Kruse et al., 2014; Lane et al., 2017; Matson et al., 2014). Mad1:GFP overexpression did not affect development of embryos to the blastocyst stage. Mad1:GFP displayed expected spatiotemporal dynamics, accumulating at the nuclear periphery in interphase, then on kinetochores shortly after nuclear breakdown, leaving kinetochores as chromosomes align (Emre et al., 2011; Lane et al., 2017; Howell et al., 2004) (FIGS. 8A-B). The inventors performed live imaging of H2B:RFP and Mad1:EGFP-expressing embryos in 500 nM GSK923295, again observing multiple misaligned chromosomes at anaphase onset (mean 3.5±0.4 per cell). Notably, 80% of misaligned chromosomes at anaphase onset clearly harboured Mad1:GFP at kinetochores (FIGS. 2F and G). Together these data suggest that misaligned chromosomes mount a SAC signal, but that SAC signalling from a moderate number of kinetochores (at least up to nine) cannot usually prevent anaphase onset.

### Example 4: Sac Strength is not Governed by Cell Size in Mouse Embryos

Recent work in mouse oocytes and C. elegans embryos has revived the longstanding notion that SAC efficiency could be affected by cell size (Chen et al., 2016; Hara et al., 1980; Minshull et al., 1994; Zhang et al., 2015). Specifically, it is thought that diffusible MCC may fail to sufficiently inhibit APC/C throughout the cytoplasm in very large cells (Kyogoku et al., 2017; Lane et al., 2017; Galli et al., 2016). Preimplantation mitotic divisions are unaccompanied by cell growth, causing cells to decrease in size ˜40-fold from ˜200 pL 1-cell stage, similar to the oocyte, to ˜5 pL at blastocyst stage (Courtois et al., 2012; Tsichlaki et al., 2016). The inventors tested whether failure of SAC to arrest mitosis may relate to the cytoplasmic volume of early preimplantation blastomeres, and that SAC efficiency might strengthen as blastomeres decrease in size during development. They tested this hypothesis with two series of experiments.

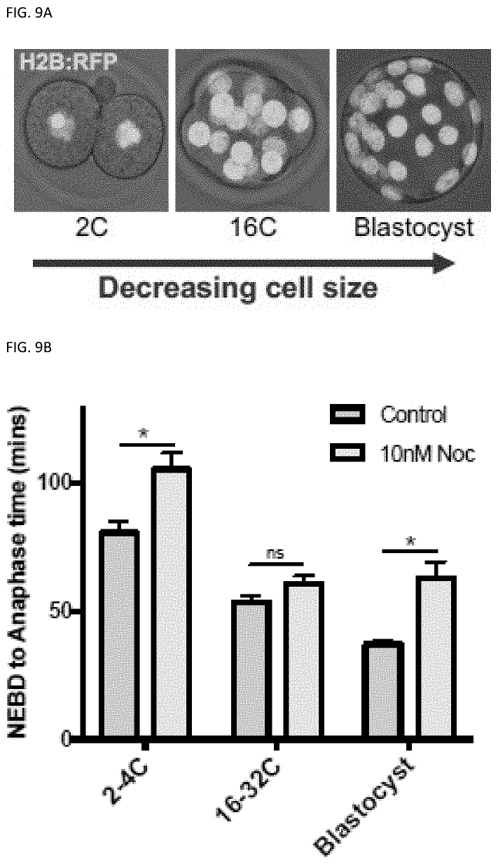

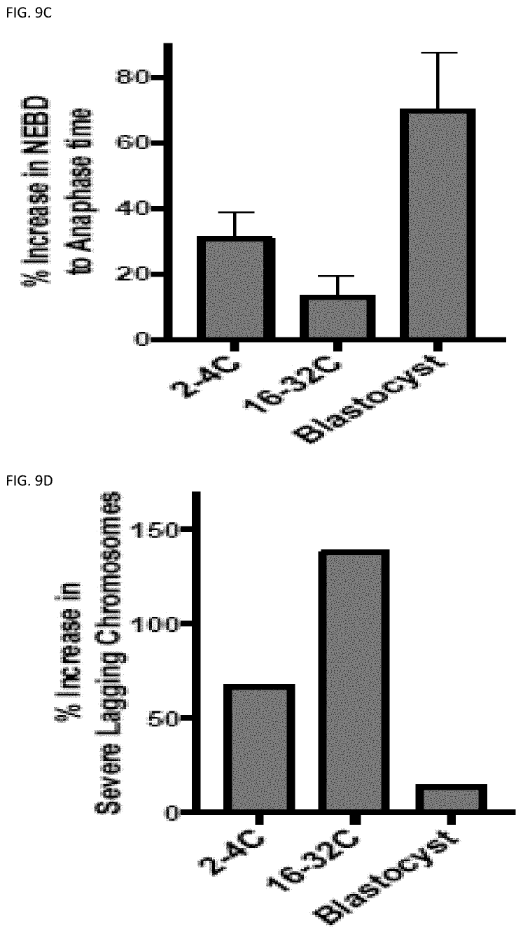

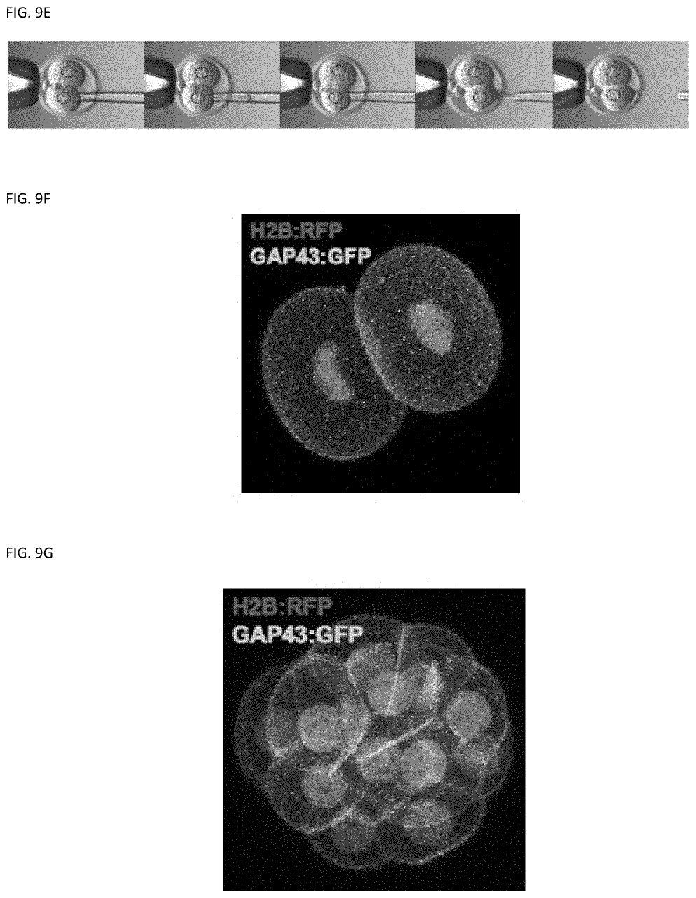

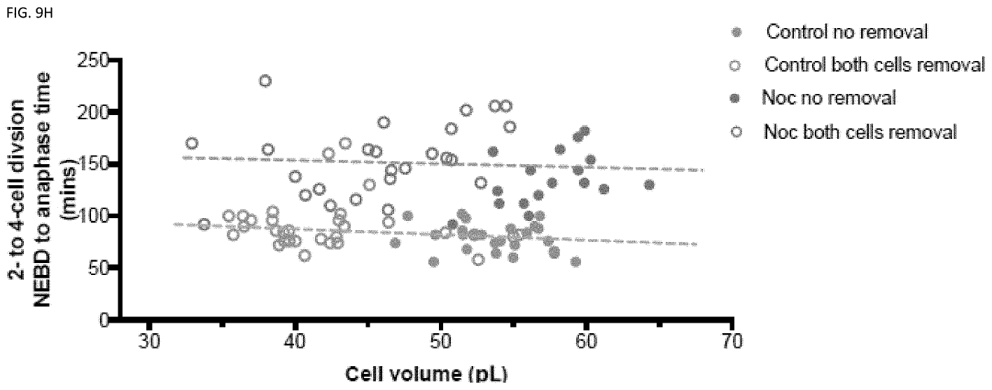

Firstly, the inventors examined SAC strength throughout preimplantation development. They performed live imaging of H2B:RFP-expressing embryos in the presence and absence of a mild spindle challenge (10 nM nocodazole) and used the increase in mitotic duration as a quantitative measure of SAC strength (Lane et al., 2017; Galli et al., 2016). Strikingly, nocodazole prolonged mitosis appreciably at the two-cell stage and in blastocysts (˜32-64 cell stage, FIGS. 9A and B). However, nocodazole treatment induced only a minimal extension of mitosis in morulae (8-16 cell stage). Anaphase lagging chromosomes were most common in nocodazole-treated morulae, which confirms the effectiveness of nocodazole at this experimental stage and is consistent with a failure of the SAC (19% of divisions, FIGS. 9C and D). Thus, SAC efficiency varies during development, but is at its weakest in morula, where cells are smaller than two-cell but larger than blastocyst-stage.

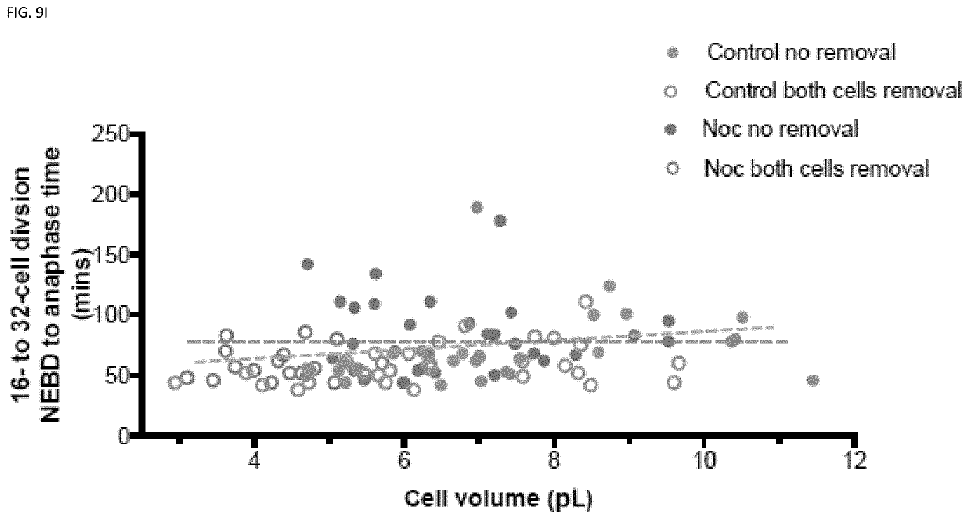

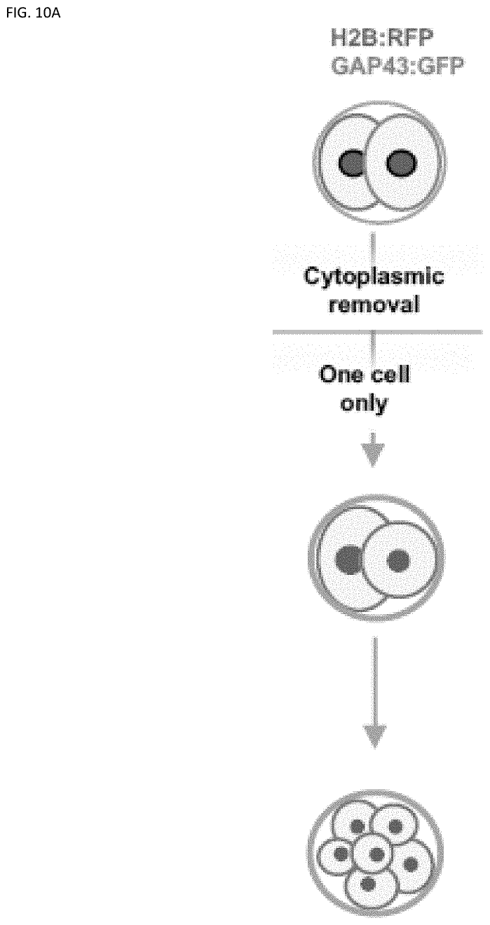

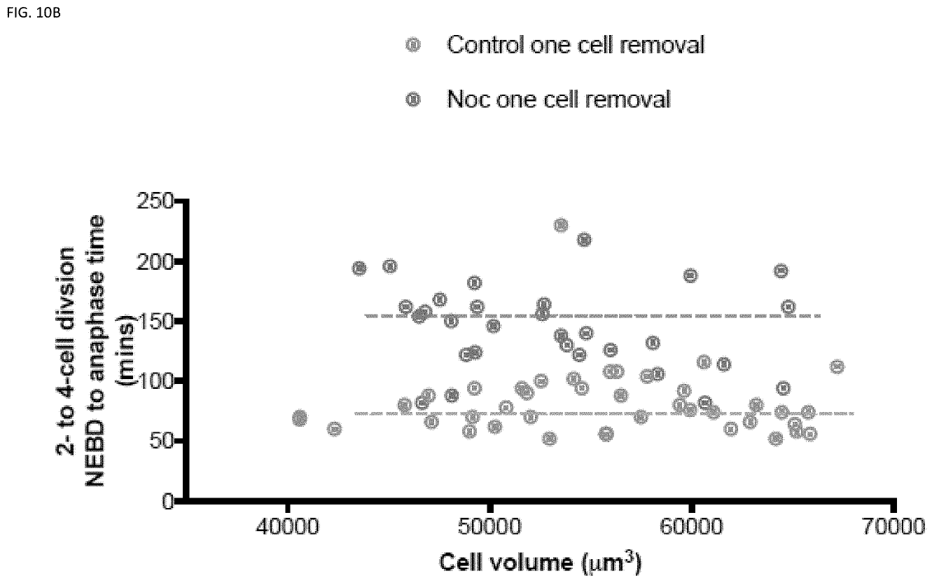

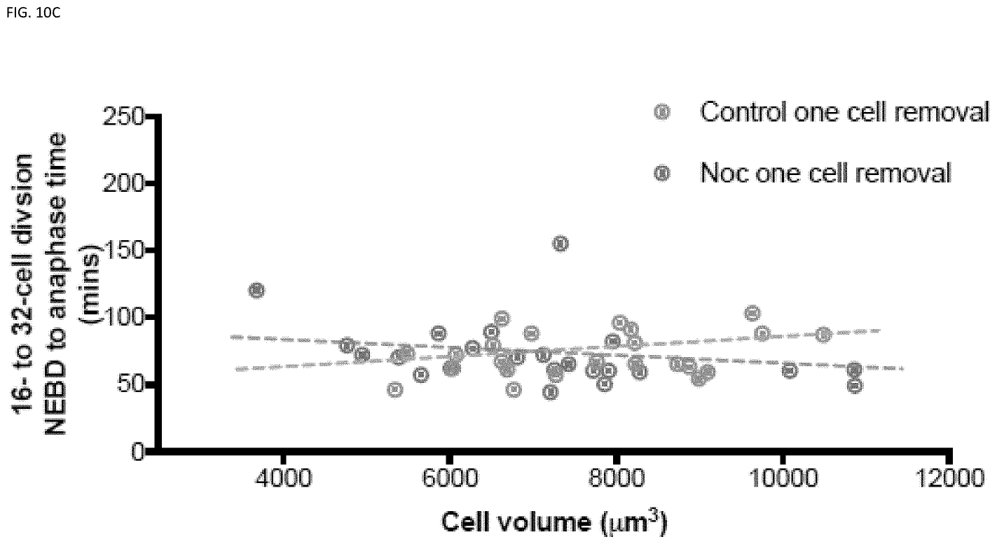

Secondly, the inventors used embryo micromanipulation to directly alter cell size (FIG. 9E). Up to 40% of cytoplasm was removed from interphase two-cell embryos using a cytoplasm-aspiration pipette. Mitosis was then examined in the presence and absence of nocodazole at the 2-4 cell transition and in morulae (FIGS. 9F and G). Importantly, embryos co-expressed H2B:RFP (chromosomes) and GAP43:GFP (plasmalemma) during live imaging, allowing mitosis duration and cell volume to be simultaneously analysed on a cell-by-cell basis. Blastomeres in cytoplasmically-reduced morulae were as small as 3 pL in volume, smaller than typical cultured cells, ˜3-5 pL (Zlotek-Zlotkiewicz et al., 2015). As in the previous experiment (FIG. 9B), nocodazole extended mitosis at the two-cell stage but not in morulae (FIGS. 9H and I). Regression analysis showed no association between cell size and mitosis duration at either developmental stage, regardless of the presence or absence of nocodazole. Similar results were obtained in a separate series of experiments in which only a single blastomere was manipulated per embryo (FIGS. 10A-C). The inventors conclude that the inability of SAC to prevent anaphase in preimplantation embryos is not directly attributable to cell size.

### Example 5: Partial APC/C Inhibition can Reduce Chromosome Segregation Errors

The inventors tested whether low concentrations of proTAME would improve chromosome segregation outcomes in normally-cultured embryos.

ProTAME is a is a cell permeable prodrug that is converted to TAME (Tosyl-L-Arginine Methyl Ester) by intracellular esterases. TAME structurally mimics the IR-tail of the co-activators and therefore binds to APC/C, blocking the interaction of Cdc20 or Cdh1 with the APC/C.

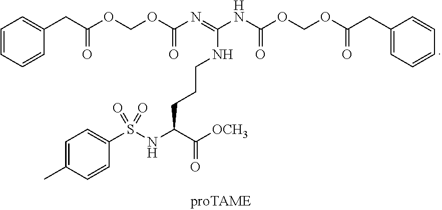 [SMILES: COC(=O)[C@H](CCCN/C(=N\C(=O)OCOC(=O)Cc1ccccc1)NC(=O)OCOC(=O)Cc1ccccc1)NS(=O)(=O)c1ccc(C)cc1]

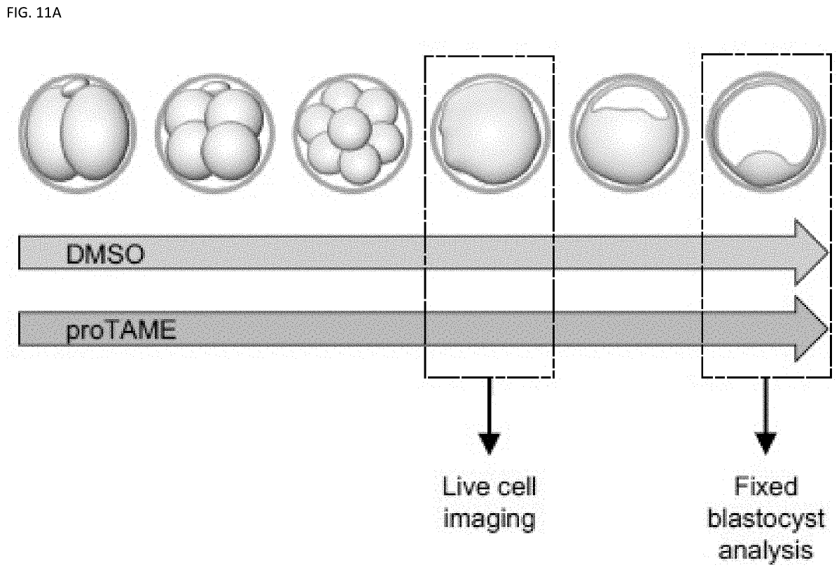

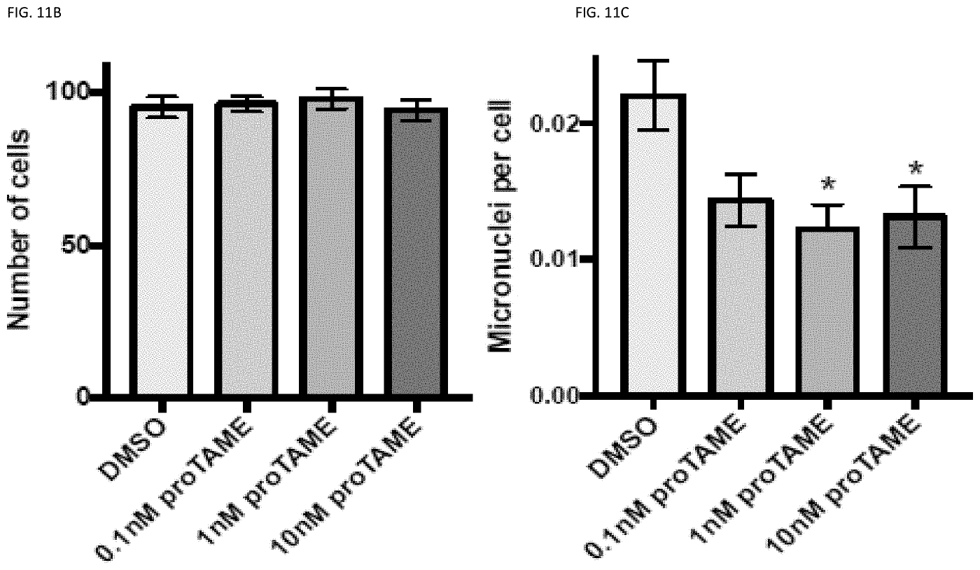

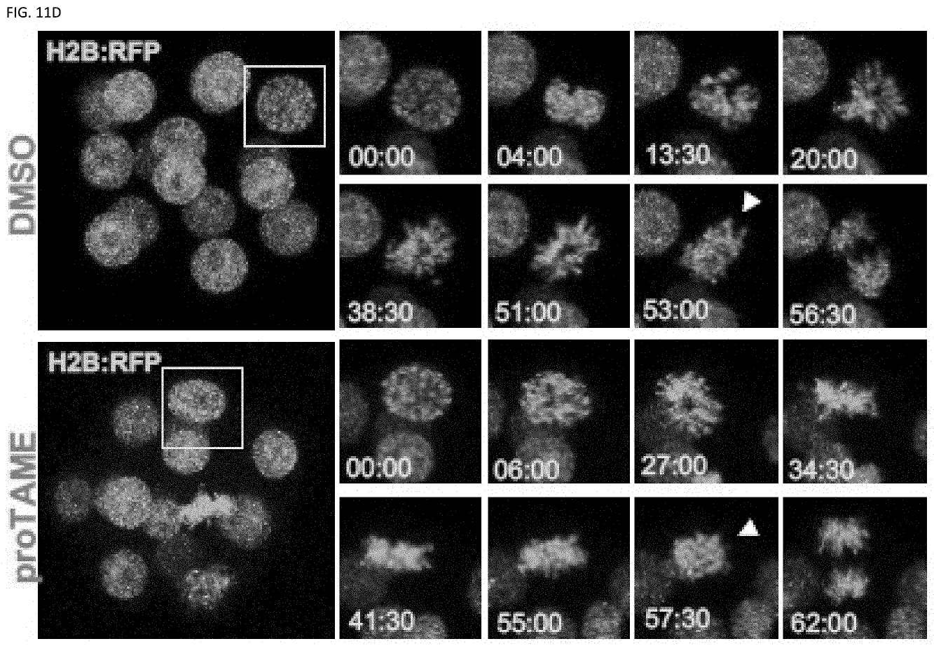

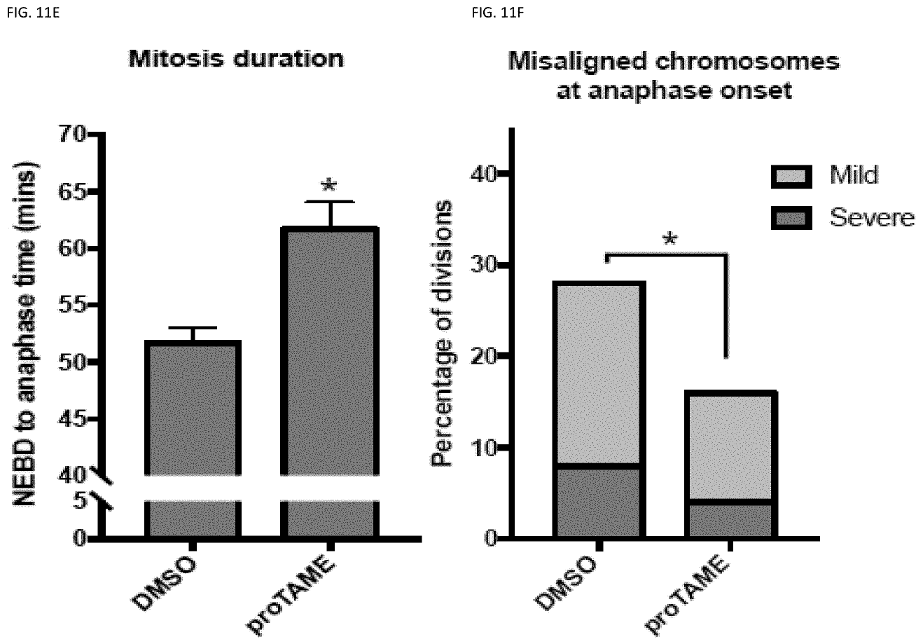

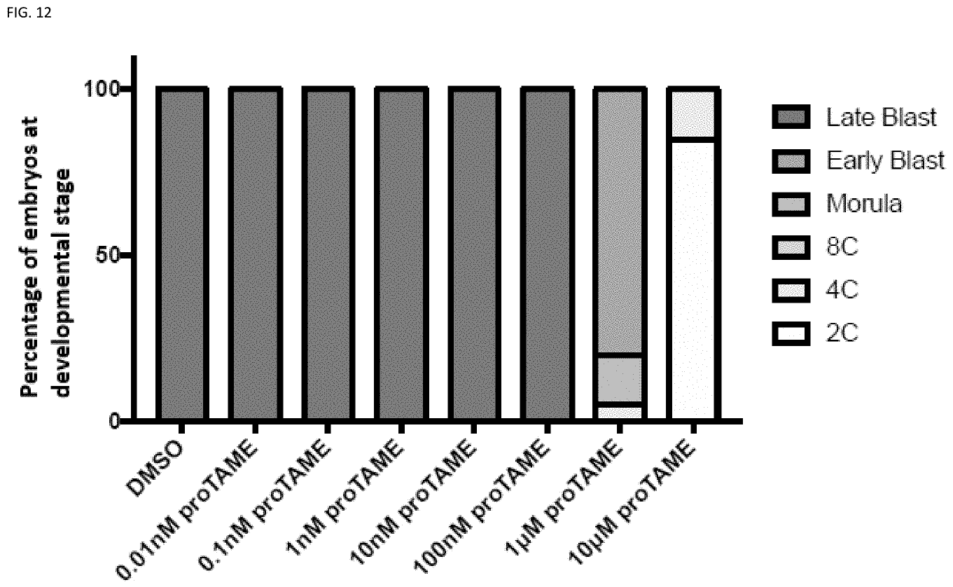

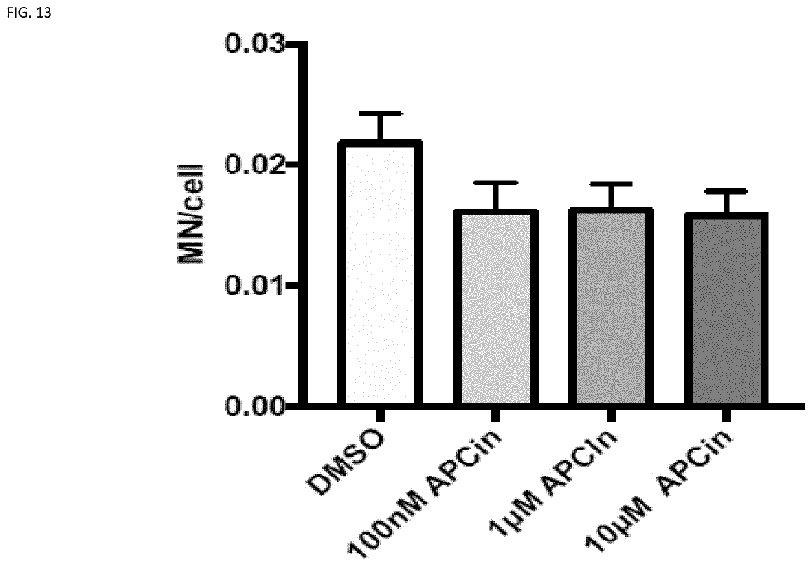

Embryos were cultured in various concentrations of proTAME from the 2-cell stage onwards. Micromolar concentrations (1-10 μM) of proTAME prevented embryo development to blastocyst (FIG. 11A, FIG. 12). In contrast, nanomolar proTAME (0.01 nM-100 nM) was permissive of development to blastocyst (FIG. 11A, FIG. 12) and did not affect blastocyst cell number (FIG. 11B). Strikingly, embryos cultured from the 2-cell stage to blastocyst in nM and 10 nM proTAME exhibited a substantial reduction in micronuclei, from 0.022 micronuclei per cell in controls to 0.012 and 0.013, respectively (P<0.05) (FIG. 11C). Live imaging revealed that 10 nM proTAME increased the duration of mitosis by ˜10 mins (from 51.7±1.4 to 61.7±2.3 min, 19% extension, P<0.0001) and reduced the incidence of misaligned chromosomes at anaphase onset from 28% to 16% (P=0.025) (FIGS. 11E and 11 F). A reduction in micronuclei was also observed in experiments employing low concentrations of APCin (10 μM), a second APC/C inhibitor which acts by binding the mitotic APC/C cofactor Cdc20 (Sackton et al., 2014) (FIG. 13). Hence, mild pharmacological APC/C inhibition extends mitosis, allowing time for more chromosomes to align before anaphase, and reduces the incidence of segregation error in embryos. To the inventors' knowledge these studies provide the first example of a specific chemical intervention improving ploidy outcomes in cultured embryos.

The scope of the claims should not be limited by the embodiments set forth in the examples but should be given the broadest interpretation consistent with the description as a whole.

### REFERENCES

Bennett, A., Bechi, B., Tighe, A., Thompson, S., Procter, D. J., and Taylor, S. S. (2015). Cenp-E inhibitor GSK923295: Novel synthetic route and use as a tool to generate aneuploidy. Oncotarget 6, 20921-20932.
Bolton, H., Graham, S. J. L., Van der Aa, N., Kumar, P., Theunis, K., Fernandez Gallardo, E., Voet, T., and Zernicka-Goetz, M. (2016). Mouse model of chromosome mosaicism reveals lineage-specific depletion of aneuploid cells and normal developmental potential. Nature Communications 7, 11165.
Brison, D. R., Roberts, S. A., and Kimber, S. J. (2013). How should we assess the safety of IVF technologies? Reprod. Biomed. Online 27, 710-721.
Brito, D. A., Yang, Z., and Rieder, C. L. (2008). Microtubules do not promote mitotic slippage when the spindle assembly checkpoint cannot be satisfied. The Journal of Cell Biology 182, 623-629.
Chen, J., and Liu, J. (2016). Spindle Size Scaling Contributes to Robust Silencing of Mitotic Spindle Assembly Checkpoint. Biophysical Journal 111, 1064-1077.
Chen, R. H., Waters, J. C., Salmon, E. D., and Murray, A. W. (1996). Association of spindle assembly checkpoint component XMAD2 with unattached kinetochores. Science 274, 242-246.
Courtois, A., Schuh, M., Ellenberg, J., and Hiiragi, T. (2012). The transition from meiotic to mitotic spindle assembly is gradual during early mammalian development. J. Cell Biol. 198, 357-370.
Echten-Arends, J. van, Mastenbroek, S., Sikkema-Raddatz, B., Korevaar, J. C., Heineman, M. J., Veen, F. van der, and Repping, S. (2011). Chromosomal mosaicism in human preimplantation embryos: a systematic review. Hum. Reprod. Update 17, 620-627.
Emre, D., Terracol, R., Poncet, A., Rahmani, Z., and Karess, R. E. (2011). A mitotic role for Mad1 beyond the spindle checkpoint. J. Cell. Sci. 124, 1664-1671.
Galli, M., and Morgan, D. O. (2016). Cell Size Determines the Strength of the Spindle Assembly Checkpoint during Embryonic Development. Dev. Cell 36, 344-352.
Gerhold, A. R., Poupart, V., Labbe, J.-C., and Maddox, P. S. (2018). Spindle assembly checkpoint strength is linked to cell fate in the C. elegans embryo. Mol. Biol. Cell mbcE18040215.
Gui, L., and Homer, H. (2012). Spindle assembly checkpoint signalling is uncoupled from chromosomal position in mouse oocytes. Development 139, 1941-1946.
Guo, G., Huss, M., Tong, G. Q., Wang, C., Li Sun, L., Clarke, N. D., and Robson, P. (2010). Resolution of Cell Fate Decisions Revealed by Single-Cell Gene Expression Analysis from Zygote to Blastocyst. Developmental Cell 18, 675-685.
Hara, K., Tydeman, P., and Kirschner, M. (1980). A cytoplasmic clock with the same period as the division cycle in Xenopus eggs. Proc Natl Acad Sci USA 77, 462-466.
Heinrich, S., Sewart, K., Windecker, H., Langegger, M., Schmidt, N., Hustedt, N., and Hauf, S. (2014). Mad1 contribution to spindle assembly checkpoint signalling goes beyond presenting Mad2 at kinetochores. EMBO Reports 15, 291-298.
Hewitt, L., Tighe, A., Santaguida, S., White, A. M., Jones, C. D., Musacchio, A., Green, S., and Taylor, S. S. (2010). Sustained Mps1 activity is required in mitosis to recruit O-Mad2 to the Mad1-C-Mad2 core complex. The Journal of Cell Biology 190, 25-34.
Homer, H. A., McDougall, A., Levasseur, M., Yallop, K., Murdoch, A. P., and Herbert, M. (2005). Mad2 prevents aneuploidy and premature proteolysis of cyclin B and securin during meiosis I in mouse oocytes. Genes Dev. 19, 202-207.
Howell, B. J., Moree, B., Farrar, E. M., Stewart, S., Fang, G., and Salmon, E. D. (2004). Spindle checkpoint protein dynamics at kinetochores in living cells. Curr. Biol. 14, 953-964.
Kolano, A., Brunet, S., Silk, A. D., Cleveland, D. W., and Verlhac, M.-H. (2012). Error-prone mammalian female meiosis from silencing the spindle assembly checkpoint without normal interkinetochore tension. Proc. Natl. Acad. Sci. U.S.A. 109, E1858-1867.
Kruse, T., Larsen, M. S. Y., Sedgwick, G. G., Sigurdsson, J. O., Streicher, W., Olsen, J. V., and Nilsson, J. (2014). A direct role of Mad1 in the spindle assembly checkpoint beyond Mad2 kinetochore recruitment. EMBO Reports 15, 282-290.
Kyogoku, H., and Kitajima, T. S. (2017). Large Cytoplasm Is Linked to the Error-Prone Nature of Oocytes. Dev. Cell 41, 287-298.e4.
Lane S I, Yun Y, Jones K T. Timing of anaphase-promoting complex activation in mouse oocytes is predicted by microtubule-kinetochore attachment but not by bivalent alignment or tension. Development. 2012 June; 139(11):1947-55.
Lane, S. I. R., and Jones, K. T. (2017). Chromosome biorientation and APC activity remain uncoupled in oocytes with reduced volume. J. Cell Biol.
Lane, S. I. R., Morgan, S. L., Wu, T., Collins, J. K., Merriman, J. A., Ellnati, E., Turner, J. M., and Jones, K. T. (2017). DNA damage induces a kinetochore-based ATM/ATR-independent SAC arrest unique to the first meiotic division in mouse oocytes. Development 144, 3475-3486.
Lara-Gonzalez, P., Westhorpe, F. G., and Taylor, S. S. (2012). The Spindle Assembly Checkpoint. Current Biology 22, R966-R980.
Lee, M. T., Bonneau, A. R., and Giraldez, A. J. (2014). Zygotic genome activation during the maternal-to-zygotic transition. Annu. Rev. Cell Dev. Biol. 30, 581-613.
Li, X., and Nicklas, R. B. (1995). Mitotic forces control a cell-cycle checkpoint. Nature 373, 630-632.
Mantikou, E.; Youssef, M. A. F. M.; Van Wely, M.; Van Der Veen, F.; Al-Inany, H. G.; Repping, S.; Mastenbroek, S. (2013). “Embryo culture media and IVF/ICSI success rates: A systematic review”. Human Reproduction Update, 19 (3): 210-220.
Matson, D. R., and Stukenberg, P. T. (2014). CENP-I and Aurora B act as a molecular switch that ties RZZ/Mad1 recruitment to kinetochore attachment status. J Cell Biol 205, 541-554.
McCoy, R. C. (2017). Mosaicism in Preimplantation Human Embryos: When Chromosomal Abnormalities Are the Norm. Trends in Genetics 33, 448-463.
Meraldi, P., Draviam, V. M., and Sorger, P. K. (2004). Timing and Checkpoints in the Regulation of Mitotic Progression. Developmental Cell 7, 45-60.
Minshull, J., Sun, H., Tonks, N. K., and Murray, A. W. (1994). A MAP kinase-dependent spindle assembly checkpoint in Xenopus egg extracts. Cell 79, 475-486.
Miyanari, Y., Ziegler-Birling, C., and Torres-Padilla, M.-E. (2013). Live visualization of chromatin dynamics with fluorescent TALEs. Nat. Struct. Mol. Biol. 20, 1321-1324.
Musacchio, A. (2015). The Molecular Biology of Spindle Assembly Checkpoint Signaling Dynamics. Curr. Biol. 25, R1002-1018.
Nagaoka, S. I., Hassold, T. J., and Hunt, P. A. (2012). Human aneuploidy: mechanisms and new insights into an age-old problem. Nat Rev Genet 13, 493-504.
Nakagawa, S., and FitzHarris, G. (2016). Quantitative Microinjection of Morpholino Antisense Oligonucleotides into Mouse Oocytes to Examine Gene Function in Meiosis-I. Methods Mol. Biol. 1457, 217-230.
Nakagawa, S., and FitzHarris, G. (2017). Intrinsically Defective Microtubule Dynamics Contribute to Age-Related Chromosome Segregation Errors in Mouse Oocyte Meiosis-I. Curr. Biol. 27, 1040-1047.
Petropoulos, S., Edsgärd, D., Reinius, B., Deng, Q., Panula, S. P., Codeluppi, S., Plaza Reyes, A., Linnarsson, S., Sandberg, R., and Lanner, F. (2016). Single-Cell RNA-Seq Reveals Lineage and X Chromosome Dynamics in Human Preimplantation Embryos. Cell 165, 1012-1026.
Rieder, C. L., Cole, R. W., Khodjakov, A., and Sluder, G. (1995). The checkpoint delaying anaphase in response to chromosome monoorientation is mediated by an inhibitory signal produced by unattached kinetochores. The Journal of Cell Biology 130, 941-948.
Sackton, K. L., Dimova, N., Zeng, X., Tian, W., Zhang, M., Sackton, T. B., Meaders, J., Pfaff, K. L., Sigoillot, F., Yu, H., et al. (2014). Synergistic blockade of mitotic exit by two chemical inhibitors of the APC/C. Nature 514, 646-649.
Sansregret, L., Patterson, J. O., Dewhurst, S., López-Garcia, C., Koch, A., McGranahan, N., Chao, W. C. H., Barry, D. J., Rowan, A., Instrell, R., et al. (2017). APC/C Dysfunction Limits Excessive Cancer Chromosomal Instability. Cancer Discov 7, 218-233.
Taylor, T. H., Gitlin, S. A., Patrick, J. L., Crain, J. L., Wilson, J. M., and Griffin, D. K. (2014). The origin, mechanisms, incidence and clinical consequences of chromosomal mosaicism in humans. Hum Reprod Update 20, 571-581.
Tsichlaki, E., and FitzHarris, G. (2016). Nucleus downscaling in mouse embryos is regulated by cooperative developmental and geometric programs. Sci Rep 6, 28040.
Vanneste, E., Voet, T., Le Caignec, C., Ampe, M., Konings, P., Melotte, C., Debrock, S., Amyere, M., Vikkula, M., Schuit, F., et al. (2009). Chromosome instability is common in human cleavage-stage embryos. Nat Med 15, 577-583.
Vázquez-Diez, C., and FitzHarris, G. (2018). Causes and consequences of chromosome segregation error in preimplantation embryos. Reproduction 155, R63-R76.
Vázquez-Diez, C., and FitzHarris, G. (2018). Correlative Live Imaging and Immunofluorescence for Analysis of Chromosome Segregation in Mouse Preimplantation Embryos. Methods Mol. Biol. 1769, 319-335.
Vázquez-Diez, C., Yamagata, K., Trivedi, S., Haverfield, J., and FitzHarris, G. (2016). Micronucleus formation causes perpetual unilateral chromosome inheritance in mouse embryos. PNAS 201517628.
Ventura-Juncá, P., Irarrázaval, I., Rolle, A. J., Gutiérrez, J. I., Moreno, R. D., and Santos, M. J. (2015). In vitro fertilization (IVF) in mammals: epigenetic and developmental alterations. Scientific and bioethical implications for IVF in humans. Biol. Res. 48, 68.
Wassmann, K., Niault, T., and Maro, B. (2003). Metaphase I arrest upon activation of the Mad2-dependent spindle checkpoint in mouse oocytes. Curr. Biol. 13, 1596-1608.
Wei, Y., Multi, S., Yang, C.-R., Ma, J., Zhang, Q.-H., Wang, Z.-B., Li, M., Wei, L., Ge, Z.-J., Zhang, C.-H., et al. (2011). Spindle Assembly Checkpoint Regulates Mitotic Cell Cycle Progression during Preimplantation Embryo Development. PLoS ONE 6, e21557.
Wells, D., Bermudez, M. G., Steuerwald, N., Thornhill, A. R., Walker, D. L., Malter, H., Delhanty, J. D. A., and Cohen, J. (2005). Expression of genes regulating chromosome segregation, the cell cycle and apoptosis during human preimplantation development. Hum. Reprod. 20, 1339-1348.
Young, L. E., and Fairburn, H. R. (2000). Improving the safety of embryo technologies: possible role of genomic imprinting. Theriogenology 53, 627-648.
Zeng, X., Sigoillot, F., Gaur, S., Choi, S., Pfaff, K. L., Oh, D.-C., Hathaway, N., Dimova, N., Cuny, G. D., and King, R. W. (2010). Pharmacologic Inhibition of the Anaphase-Promoting Complex Induces A Spindle Checkpoint-Dependent Mitotic Arrest in the Absence of Spindle Damage. Cancer Cell 18, 382-395.
Zhang, M., Kothari, P., and Lampson, M. A. (2015). Spindle Assembly Checkpoint Acquisition at the Mid-Blastula Transition. PLOS ONE 10, e0119285.
Zlotek-Zlotkiewicz, E., Monnier, S., Cappello, G., Le Berre, M., and Piel, M. (2015). Optical volume and mass measurements show that mammalian cells swell during mitosis. J Cell Biol 211, 765-774.

1. Method for reducing chromosome segregation errors in cultured cells of the early embryo by contacting the cells with an effective dose of an inhibitor of the anaphase promoting complex (APC/C) that directly binds to the APC/C.

2. The method of claim 1, wherein the inhibitor is proTAME or TAME.

3. The method of claim 1, wherein the effective dose is a nanomolar amount.

4. The method of claim 1, wherein the effective dose is about 0.001 nM to about 500 nM.

5. The method of claim 1, wherein the effective dose is about 0.01 nM to about 100 nM.

6. The method of claim 1, wherein the cells are at the 2-cell stage to blastocyst stage.

7. The method of claim 1, wherein the inhibitor increases the duration of mitosis, reduces the number of micronucleis per cell, reduces the incidence misaligned chromosomes at anaphase onset or a combination thereof.

8. The method of claim 1, wherein the inhibitor does not influence blastocyst cell number as compared to cells of the early embryo not contacting with the inhibitor.

9. The method of claim 1, wherein the cells are mammalian cells.

10. The method of claim 9, wherein the cells are human cells.

11. Cells of the early embryo obtained by the method defined in claim 1.

12. Composition comprising the cells of the early embryo defined in claim 11.

13. A kit for reducing chromosome segregation errors in cultured cells of the early embryo comprising (a) an inhibitor of the anaphase promoting complex (APC/C) that directly binds to the APC/C; and (b) (i) instructions to use the inhibitor for reducing chromosome segregation errors in cultured cells of the early embryo; (ii) cells of the early embryo; and (iii) a combination of (i) and (ii).
